SEQUENCE LISTING (175 sequences)

SEQ ID NO: 1 - DNA, 660 bp, Bos taurus
  misc_feature (308)..(309): n is a, c, g, or t, as described in Table 4
cccccgcccccgcccccgcccacgctgtctcggccacgggcagcgcggggggcgtggcctgagcttgcctctcccacagtgggctccgtgctggccctgatggtctacaccatcctcttcctcaagctgttctcctaccgggacgtcaacctctggtgccgagagcgcagggctggggccaaggccaaggctggtgagggctgcctcgggctggggccactgggctgccacttgcctcgggaccggcaggggctcggctcacccccgacccgccccctgccgcttgctcgtagctttggcaggtaagnnggccaacgggggagctgcccagcgcaccgtgagctaccccgacaacctgacctaccgcggtgaggatcctgccgggggctggggggactgcccggcggcctggcctgctagccccgccctcccttccagatctctactacttcctcttcgcccccaccctgtgctacgagctcaacttcccccgctccccccgcatccgaaagcgcttcctgctgcggcgactcctggagatggtgaggcggggcctcgcgggccagggtgggcgggcctgccggcacccggcaccggggctcagctcactgtccgcttgcttccttccccagctgttcctcacccagctccaggtggggc

SEQ ID NO: 2 - DNA, 720 bp, Bos taurus
  misc_feature (307)..(307): n is a, c, g, t, or a deletion, as described in Table 4
taaataggagctgacatcctacatagggccatttataataaataggctattataataaatagggccatttatctttactctcaccttttgcatgattcttacaatggaagcgtgagataaatgaatagtgcaatctccatttcacaactgagaaaggtagatgaagaggttaagtaatcttgaaacaatattaaatgtttaaaatgaactcagagctctgctacccctaacttctgttccaatattcaaccttcatccataattttctttcaaacaccttttaaatgcccattaaagtttttttttnaatatagaatttttattttcttattcagtaaccaattttatatatcctgagagaaaaattagaaaatgacaattaagaaatctaagccagtccttcagagacatgcaaattatcctgttgacatacagtataaaaatcttatatccgatctcattacaataaaccattccatttagagttaatacaaatcatgactacctttttctcctaaaaatcttaataattgttaacatacaattaaatatggttaaaatatgcagggtatttgcaaatatgtgggaggtatttttagttttacacattctaattcacttaaatctctcaaaaaccccacgaactctgcatttgacagatgaagaaacaagtatagataggctaaatgatttgcccaaggtcacacacctaatttgtgc

SEQ ID NO: 3 - DNA, 180 bp, Bos taurus
  misc_feature (63)..(63): n is a, c, g, or t, as described in Table 4
atataccaccatttggctcatcagtccaacaccagcaacatcttcttctcccccgtgagcatngcttcagcctttgcnatgctctccctgggagccaagggcaacactcacactgagatcctgaagggcctgggtttcaacctcactgagctcgcagaggctgagatccacaaaggcttt

SEQ ID NO: 4 - DNA, 480 bp, Bos taurus
  misc_feature (232)..(232): n is a, c, g, or t, as described in Table 4
ggcccttgagaggctctgcaggacaagaggatggccctgattctaatatcctctgaccctgggcatagaggaactaaaagtggaataaaccaaagtgtgagagcagggggagagggcaccaactggaaagaacaaccggaaaaggaagctctttcaactctgtgacttttttttttttcactacagttctgccaatttacatttgcccaaactgtccatttctgaaacgtangatctaaaaagtgtcctgggcgatgtgggcatcaccgaggtcttcagcgatagggctgacctctcagggatcaccaaggaacagcctctgaaggtgtccaaggtgagtgtgtccctgacgtctgtaggtcagaatgcatgcggggccacagctctggggcgaggctgaggaaggggcagagggatgcaggcacgccagcagaccaaggcccctgaggaatgccatcgctccacaacgacggcagtgtg

SEQ ID NO: 5 - DNA, 661 bp, Bos taurus
  misc_feature (306)..(306): n is a, c, g, or t, as described in Table 4
tggttgttttgcttttaataattatctattaaagaaaggaaggatattgtactatatgtttgtgaggtcagaaattgttagcattgaccatgatttataattacatggccactaaaaaggttggacaacaactaaatgttcagcataggaaattagtaagttattgaaaatcacatagcagcaatatgcacacattaaaaattatgttgtaaagtaatatttaatgatgtaggaaaataactttattttgtgagctggaaagaaccggattataaaatggtatgtgttttctgatcacacacattncaatcaatacacacactcacaccaaaatatacattatcactattgggagtaggatcaggaatctttaagctcttctttgtgcttttctgcttttcataaaaacatctacaggggacttccctggtggtccagtggctaagactccctgctttcaaatgcaggggccccaggttcgatatctggtcagggaactgaactgggtcccgcatgccgcagctaagagttctcatgctgcgactaaacatcctgcctgctgaaactaaggcctggaactgtcaaataaagaaatgtttcttttaaagaagtgtctacaatgaaattacattttgaagaaaatctttcctcctctccgcct

SEQ ID NO: 6 - DNA, 420 bp, Bos taurus
  misc_feature (240)..(240): n is a, c, g, or t, as described in Table 4
tgcccccaaatgagaacaaattattggcatataactttaagaatagcataaatgtgtacatttgaaatgaaacgaatgtgtcttgaatcctcatacattttcttaccagtcccgtctattttgtctttgatccaaactcctaaatgtttgtgcacatgttttgtggtgacaatgctgggaaacacagcaacaggacttcattattctgttccttcctgtcattatggaaaccagtcatcnacctatggcgtgatggcaggtaagaaaaattgtctttacatgtaagattgagtttggggacgcttggatgcattttctgggtcgaagggaatcttgaccagagtgtatcatgaaattcagatctcctaaccttagaaattgctgctaaatccaccacttactataatggtccctgatctg

SEQ ID NO: 7 - DNA, 300 bp, Bos taurus
  misc_feature (198)..(198): n is a, c, g, or t, as described in Table 4
gtcagtggccaagggactcactgtatggtctgatccaggcatggtacccttctctcttgcaggataatggcactctccatcacgcggggccttctgctgctggcagccctgtgctgcctggcccccatctccctggctggagttctccaaggacacgctgtccaagagacagatgatacatcccaccaggaagcagcntgccacaagattgcccccaacctggccaactttgccttcagcatataccaccatttggctcatcagtccaacaccagcaacatcttcttctcccccgtgagc

SEQ ID NO: 8 - DNA, 480 bp, Bos taurus
  misc_feature (244)..(244): n is a, c, g, or t, as described in Table 4
agtggatagaggtgttcttagaaaatactaagtaattgcattctatttcagtggctatcaagtgaaatcattgactttactagatgaatacaaattaggaagttttatgtggaacaggagaatgagatataaacttcaactgttcatagttctgtgagatattatttttgtgtttttcagatttccagtttccatggttcttaattattatctttggaatacttgggctagcagtgacattatntttactcatattttctaaacagcaaaggtaagtgtgatataacctactctgatatgttttgccagttatttagcaaatgtccatgtttccattttttgtttgatgttttcttttgtgaatcctgagtgaagtgtttcatcaacccagtgaaacgttatcgctctacatttacatctttgttgtgtccacagagagacaacacaggtctcagttttatctggaaagttgcataggat

SEQ ID NO: 9 - DNA, 720 bp, Bos taurus
  misc_feature (365)..(365): n is a, c, g, or t, as described in Table 4
ctgtgccattcaatgggtagctcataggaaatcaaagaaaagctatggcatgattttgttcagttggtctgtgctcacatagccacatgatgagagaaactctttgtcaggcaagggcagggcagtcgcattgagtacgaggccctgtggagactgtactatatgaatggaggtataatctgggacaggtatctcagaacttggaacatgttctgctgtccccgacctcccagctgtagtggtaaggctttctgtggtggtgtaaatgtcttcctggttaaagcttggctctacgtgtgattcagcctcgacatgaggggccagggcaatgtactttttggcgtctacctcgcagaagtaagcgntgtccacgatgaagttagcttggcagggtgtgaccacttctgggtgcgtgtcacactgggggttcccagtcttattcttttggcctggggaaaggaccacatttcctgctggtgtaatgtcgcttacctgggcataaaaatcaatgtttgccaatgaacttggattgctgagctgtgtatggacagcttgatgagttgactcagttccaccaatgagaagtggtcttggtttgttttcctctactaggataacactgggctgctggctagcaggggcagcatcattagaaggtgaattattttgattcttctgatcaaggcatgagatatctgcttccccttttaacctttgtgg

SEQ ID NO: 10 - DNA, 346 bp, Bos taurus
  misc_feature (81)..(81): n is a, c, g, or t, as described in Table 4
ctgaagggcctgggtttcaacctcactgagctcgcagaggctgagatccacaaaggctttcagcatcttctccacaccctnaaccagccaaaccaccagctgcaactgaccactggcaatggtctgttcatcaatgagagtgcaaagctagtggatacgtttttggaggatgtcaagaacctgtatcactccgaagccttctccatcaacttcagggatgctgaggaggccaagaagaagatcaacgattatgtagagaagggaagccatggaaaaattgtggagttggtaaaggttcttgacccaaacacagtttttgctctggtgaattacatttcctttaaag

SEQ ID NO: 11 - DNA, 480 bp, Bos taurus
  misc_feature (247)..(247): n is a, c, g, or t, as described in Table 4
ggagagtgagaaggcgggaggcaagggaagtggaggaggagaggagctgtctttatgccaggggggcgtccaggccactggggccctgtgcaaggctgcacagcctcctccgccagcctctggggtcccccacgggatggccacggttctacctcgtctcccagtccctccctaccgtgtgtgagatgtcattgatcctggtgacaattgtcttgatgagggttttggtgtcatcctggaccttgcngatgggcacagcctccacgtaagacagatagggccaaagccacaggaatcgatacaggggtccacagcgcattttccttcccgggatgggcttctggggcctgaaaacagaagaaaccacacgtggcacatcgtcgatctccgagaacacccacgtgctccgttaccacccgcatccaggtcttcagatgcggataacaacaagatttgctgtctgccatggctatcatctcc

SEQ ID NO: 12 - DNA, 180 bp, Bos taurus; identical to SEQ ID NO: 3
  misc_feature (63)..(63): n is a, c, g, or t, as described in Table 4

SEQ ID NO: 13 - DNA, 360 bp, Bos taurus
  misc_feature (214)..(214): n is a, c, g, or t, as described in Table 4
atggtgtatccttccatggatattctttttctgtccttcatatatccatttccttaaaaaaaaaagtgtgcaggccttccttggtggtacagtggatgagaatccgcctgccagtgcagggacatgggatcggtccctggttgaggaagattccacatgctgggagcaacaaaggccgtgtgacacggctcccgagcccaagctctagagcctntgtgttgcaaccgctgagtccctgggcacctggagcctatgctccacaacaggagaagctgccacagtgagaagcttgcacattgcaatgaagacccagcatagcaaaaaataaataaattaattaaaaatatatatatttaaggg

SEQ ID NO: 14 - DNA, 69 bp, Bos taurus
  misc_feature (37)..(37): n is a, c, g, or t, as described in Table 4
ccgaagcagtagagagcactgtagctactctagaagnttctccagaagttattgagagcccacctgaga

SEQ ID NO: 15 - DNA, 2356 bp, Bos taurus
  misc_feature (7)..(7): n is a, c, g, or t
ttacagnagtgagtcatttgtactacaattcctcccttgtgaatcantgctatcactgggcacagtaccttgctggttcacaaagttttcggtgcctggggggtcttcacacagnagttcactgtctttctcctcccccaggtgatatccactctgttcccctgtgtagtcagtcctgtcattgctgttgctggagcagccattcccataggccttcaatgaagacctgcncaggcagagaagctcctggaaggcaatcctgaaatctgggctccggcagtagataaggggattgaaagcngagttgatgtagcccaaccagtttagaaggatntatatttccttacggatgaggttatccttgatcacgtgcacaatgttgacaatgaagaagggcagccagcacagggtgaaagtgcccatgataatgcctaaagtcttgagggctttgtgttccttcaagtagaacttggaggtcctgcgttgtcctagaccgctccgcccatcctgctccacttgactgacgttttgggcatggaagcggccctcagatttgtcnatcttctggagctgccttttggccacctggaacaccctggagtagacgaagaccatgaccaccaggggaaggtagaaggacacaatggaggaggcaatggcatagggttggttcgtgaagaagtcacagcaggtttccttagcatagcagttgatggcttccttgtggctggcccggtaccagtgcatctgaatgggtaagaaggaggtaaggccagacacgatccacacnatcaaaatgaccacccgggccttattcttggtcagcaggcactgatacttgaagggtgacgtgatggctaagtagcgatccacagcgatcacgcacaaggtctcaatgctggccgtgacgcataacacgtcaatggaagtccaaaactcacaccagaagttgccaaaagtccacattttcatgaggatgtggcaggccccaaagggcaccactgccaggcccatgaccaggtcagcacaggccagggaggtgatgaagtagttggtgaccgtctggagacgctcaaacttggcaatggctgtgatgactagcacgtttccaaacacgatggccaggacnataagcgacatgaggatgcccatgcccacaacccaggcctcgtcccgttccagcgtgacgttttggtccggcgcgtggcttgcgttgngcgccagcaaaaagacgctgcggttcccgngctgccccatggcgcgcaggctggcaggtgagcgcacaggctgccggcgcaccagccgccctcagcgagcggacctccggcggggcgctgcgggnagcaagcgagcacctggaagactcattcagcggccgtgggtgggtgtgggtgtggtaggggtgcgtggtgcactcagctccggggctactctgggctcncagtgcctgtcagttcagccagttccagcttgcgctctggagaagccgtctctgagtgcgcgctgtccgttatgtgcccaggactttaggggaactgccctccccgtgacgtgctacaactttcaaccaatagaacgcgggaagccccaaaggggcgaggcccacgccctctcccgccccttccctcccttctcctgcctgctccnggggctggcccgggcggnacccaactgctctaggagggcgggtcggccaccattccctcggggctctctcggctggccccggaggctgaagccggctctggcgagcttaccagccaactagaaggtgccagttctttcntgactgctacctgtctgcctggggcgccngtgcggcttggtctcagggtagatggcaatactccggcactccctcgcattcggaaatagatgatcgtgcccaccgagacacgcacaggcaggcgcactgtaccccgacatacatgcttagacttatacggaaccacagccacagacactcagacacacccatccagcgcacnaacgagcgcgcaggcacagaagccgactggcacaaagtcacccctgtccaacacaagagacatggccaggagcaaaccaggagcacccggaagcataaagacacggacatacagacaaaaaaagtaaacaaatagcatacaaacgcccacttggaggcaagcagtgtgccacagagaaggacttccccatctggatattccaaactctttacccttgccccctggacatccactccatcttccccagtacacaggactcatggtatattccctttcagacatttggcaagaccacaggtggtgactttagcagcagccaatttcctcagcatgcttgctgtccagtacttttggtgccctcccttggg

SEQ ID NO: 16 - DNA, 2356 bp, Bos taurus
  misc_feature (369)..(369): n is a, c, g, or t
cccaagggagggcaccaaaagtactggacagcaagcatgctgaggaaattggctgctgctaaagtcaccacctgtggtcttgccaaatgtctgaaagggaatataccatgagtcctgtgtactggggaagatggagtggatgtccagggggcaagggtaaagagtttggaatatccagatggggaagtccttctctgtggcacactgcttgcctccaagtgggcgtttgtatgctatttgtttactttttttgtctgtatgtccgtgtctttatgcttccgggtgctcctggtttgctcctggccatgtctcttgtgttggacaggggtgactttgtgccagtcggcttctgtgcctgcgcgctcgttngtgcgctggatgggtgtgtctgagtgtctgtggctgtggttccgtataagtctaagcatgtatgtcggggtacagtgcgcctgcctgtgcgtgtctcggtgggcacgatcatctatttccgaatgcgagggagtgccggagtattgccatctaccctgagaccaagccgcacnggcgccccaggcagacaggtagcagtcangaaagaactggcaccttctagttggctggtaagctcgccagagccggcttcagcctccggggccagccgagagagccccgagggaatggtggccgacccgccctcctagagcagttgggtnccgcccgggccagccccnggagcaggcaggagaagggagggaaggggcgggagagggcgtgggcctcgcccctttggggcttcccgcgttctattggttgaaagttgtagcacgtcacggggagggcagttcccctaaagtcctgggcacataacggacagcgcgcactcagagacggcttctccagagcgcaagctggaactggctgaactgacaggcactgngagcccagagtagccccggagctgagtgcaccacgcacccctaccacacccacacccacccacggccgctgaatgagtcttccaggtgctcgcttgctncccgcagcgccccgccggaggtccgctcgctgagggcggctggtgcgccggcagcctgtgcgctcacctgccagcctgcgcgccatggggcagcncgggaaccgcagcgtctttttgctggcgcncaacgcaagccacgcgccggaccaaaacgtcacgctggaacgggacgaggcctgggttgtgggcatgggcatcctcatgtcgcttatngtcctggccatcgtgtttggaaacgtgctagtcatcacagccattgccaagtttgagcgtctccagacggtcaccaactacttcatcacctccctggcctgtgctgacctggtcatgggcctggcagtggtgccctttggggcctgccacatcctcatgaaaatgtggacttttggcaacttctggtgtgagttttggacttccattgacgtgttatgcgtcacggccagcattgagaccttgtgcgtgatcgctgtggatcgctacttagccatcacgtcacccttcaagtatcagtgcctgctgaccaagaataaggcccgggtggtcattttgatngtgtggatcgtgtctggccttacctccttcttacccattcagatgcactggtaccgggccagccacaaggaagccatcaactgctatgctaaggaaacctgctgtgacttcttcacgaaccaaccctatgccattgcctcctccattgtgtccttctaccttcccctggtggtcatggtcttcgtctactccagggtgttccaggtggccaaaaggcagctccagaagatngacaaatctgagggccgcttccatgcccaaaacgtcagtcaagtggagcaggatgggcggagcggtctaggacaacgcaggacctccaagttctacttgaaggaacacaaagccctcaagactttaggcattatcatgggcactttcaccctgtgctggctgcccttcttcattgtcaacattgtgcacgtgatcaaggataacctcatccgtaaggaaatatanatccttctaaactggttgggctacatcaactcngctttcaatccccttatctactgccggagcccagatttcaggattgccttccaggagcttctctgcctgngcaggtcttcattgaaggcctatgggaatggctgctccagcaacagcaatgacaggactgactacacaggggaacagagtggatatcacctgggggaggagaaagacagtgaactnctgtgtgaagaccccccaggcaccgaaaactttgtgaaccagcaaggtactgtgcccagtgatagcantgattcacaagggaggaattgtagtacaaatgactcactnctgtaa

SEQ ID NO: 17 - DNA, 382 bp, Bos taurus
  misc_feature (176)..(176): n is a, c, g, or t, as described in Table 4
catttaaataatgtcatgttattcacactaatgttcttgccccctgcctccacattttttttttggaaaaatttaaatctccacaatacacttataaggaaaaatggcattacaaatgtccatgtaccattgcccaattttaaccattattaatctcacttctttcacctagtatntctagaatacaatttcttaccaatacacaggattgtgccaatcattttagagtcagcgtatgtttcatttcacagatgcaccataatcaatctaaccataatgttagatacataatgttgttaattaatagactcaaaagtacttattggagcacagtgtgagtatatttgtgggagatttcttgataaatagaattgttgaattc

SEQ ID NO: 18 - DNA, 518 bp, Bos taurus
  misc_feature (95)..(95): n is a, c, g, or t
ctcatcaagctgtccatacacagctcagcaatccaagttcattggcaaacattgatttttatgcccaggtaagcgacattacaccagcaggaaangtggtcctttccccaggccaaaagaataagactgggaacccccagtgtgacacgcacccagaagtggtcacaccctgccaagctaacttcatcgtggacancgcttacttctgcgaggtagacnccaaaaagtacattnccctggcccctcangtcgaggctgaatcacacatagagccangctttaaccaggaagacatttacatcaccacagaaagccttaccactacagctgggaggtcggggacagcagaacatgttccaagttctgagatacctgtcccagattatacctccattcatatagtacagtctccacagggcctngtactcaatgcgactgccctgcccttgcctgacaaagagtttctctcatcatgtggctatgtgagcacagaccaactgaacaaaatcatgccatag

SEQ ID NO: 19 - DNA, 143 bp, Bos taurus
  misc_feature (72)..(72): n is a, c, g, or t, as described in Table 4
cctagaagccccgtctgatggttaggtgattaggygagacaggttatccaggaagggctcttttgggcccangtgttaagctttcctcattccagggttcccagcatagtctcagatcaatcttccatgtctgcaaaggctca

SEQ ID NO: 20 - DNA, 143 bp, Bos taurus
  misc_feature (72)..(72): n is a, c, g, or t, as described in Table 4
agtcccaccatcatggcttatcccatcatcgtggtggacctcaccaccctgatgaattccaagacttccatngcaatgtcttctcccaccatgcccaccgctcctatcactcccccaatcatggtgagtcccaccatcatggt

SEQ ID NO: 21 - DNA, 143 bp, Bos taurus
  misc_feature (72)..(72): n is a, c, g, or t, as described in Table 4
ggaggtcccgccaacacagggagcccaaccaccatggtgggtttcatcaccacgctgaggttcctgactatngtgggctccatcgccaaagtgaggcttaccaccataaggactccacgmaatctagtagccgctcccacctc

SEQ ID NO: 22 - DNA, 143 bp, Bos taurus
  misc_feature (72)..(72): n is a, c, g, or t, as described in Table 4
ggtgggtagagatggggcctggatgtctgtgtgtagcgggagcccctgaactgcccagaggtgacaaaagcngggggtgttggtgctggaagggctgtgggcctgggcacggggcctgggcagcagtctgtgacacccaggca

SEQ ID NO: 23 - DNA, 143 bp, Bos taurus
  misc_feature (72)..(72): n is a, c, g, or t, as described in Table 4
accacgctgaggttcctgactatwgtgggctccatcgccaaagtgaggcttaccaccataaggactccacgnaatctagtagccgctcccacctcagtgaggcctatcatcatggtgggcaccaccacagaaggcctacctcc

SEQ ID NO: 24 - DNA, 143 bp, Bos taurus
  misc_feature (72)..(72): n is a, c, g, or t, as described in Table 4
atcatagaggggagaattccataagaagtcaggctttcctagaagccccgtctgatggttaggtgattaggngagacaggttatccaggaagggctcttttgggcccaygtgttaagctttcctcattccagggttcccagca

SEQ ID NO: 25 - DNA, 143 bp, Bos taurus
  misc_feature (72)..(72): n is a, c, g, or t, as described in Table 4
aacctcggaaagctgccctgaagaagacgagcacgttcagaagcgcaaaggtgcgtcttgagctcacccctnacccccaactccagctgcctggcccaggttccagacctgagtcaggcctggcgtgtccttcacaaggctcg

SEQ ID NO: 26 - DNA, 143 bp, Bos taurus
  misc_feature (72)..(72): n is a, c, g, or t, as described in Table 4
gcatggagtgtaagagcctgagcccctgaaatatgctcaaatcccaagcagactggcacctgcaaggcaggntcaagccttggcttctataccagtgcaggacaagcatgcctgcccctgagtgagctgacagcaggcaggcc

SEQ ID NO: 27 - DNA, 143 bp, Bos taurus
  misc_feature (72)..(72): n is a, c, g, or t, as described in Table 4
ggaatgaattggctcagattgccctggctccgggagaccctcgccaggacatctcaaccaaccagccttctnccccatccttattaaaatcktaaacagcagatccgtgtcattgactcagcagatgtttactgggcacagtg

SEQ ID NO: 28 - DNA, 143 bp, Bos taurus
  misc_feature (72)..(72): n is a, c, g, or t, as described in Table 4
aggacctggaggtaaccggcccaacgcccccgacgcctctggaagccgctgggcctgcgctcaccaccctcngtctccgtaacgtatcgtggacaacaggaggtgcctggctcggcgaactgcagcagtggctcaagcctggg

SEQ ID NO: 29 - DNA, 143 bp, Bos taurus
  misc_feature (72)..(72): n is a, c, g, or t, as described in Table 4
tatcgtggacaacaggaggtgcctggctcggcgaactgcagcagtggctcaagcctgggctcagggtgctgnacattgcccaagcacactcgcttgcctttccgtgcgcagggctctccaccttcgaggcgctcaccacccta

SEQ ID NO: 30 - DNA, 143 bp, Bos taurus
  misc_feature (72)..(72): n is a, c, g, or t, as described in Table 4
caagtgcctaagggactgccccctaagctcagcgtgcttgatctcagctgcaacaagctaagcagggagccncggcgagacgagctgcccgaggtaaatgacctgactctggacggaaatccctttctggaccctggagccct

SEQ ID NO: 31 - DNA, 143 bp, Bos taurus
  misc_feature (72)..(72): n is a, c, g, or t, as described in Table 4
gccactgtaaaggaaagaatccacagtccagccgacaaccagagagagaggcagaggctctgagaatctacngactatggtgagagtatgttcttggggccgaagcgtgggctatttggggaaccttaggaacaggcttgggc

SEQ ID NO: 32 - DNA, 143 bp, Bos taurus
  misc_feature (72)..(72): n is a, c, g, or t, as described in Table 4
gccctggctccgggagaccctcgccaggacatctcaaccaaccagccttctrccccatccttattaaaatcntaaacagcagatccgtgtcattgactcagcagatgtttactgggcacagtgctggacagggaatccattat

SEQ ID NO: 33 - DNA, 143 bp, Bos taurus
  misc_feature (72)..(72): n is a, c, g, or t, as described in Table 4
gctgtccatgtacttctctcatggaggtgaagagttgcgtgtccatcctgctaccttctgactcccttcttnagtggatgagcatgtgctgagcgctggtttccgttgcagggctccccgacctttcagctccagaacgactg

SEQ ID NO: 34 - DNA, 143 bp, Bos taurus
  misc_feature (72)..(72): n is a, c, g, or t, as described in Table 4
gagttgtgacccccggtctcctctggccatgcaggtagtgacgtgggctgggtgcgagtcaccaggctggcngtgctgaccrccagggagatgagtctccagagccacttctgaccttgaggctcctaggatgccctagagat

SEQ ID NO: 35 - DNA, 143 bp, Bos taurus
  misc_feature (72)..(72): n is a, c, g, or t, as described in Table 4
cctcccttttcaagtctcctctgagtggcagaggtcccccagaggagtgtacccggagcatgcgggtgggancccacgggcccctgcccgccggcagggctttctgaagcccctgtgccggtctttgcgtagggtgggcgggg

SEQ ID NO: 36 - DNA, 143 bp, Bos taurus
  misc_feature (72)..(72): n is a, c, g, or t, as described in Table 4
gcgagaaggggttagtctacctcagcgtgtgcggggacaacgagaactgcgccaacggcgtgggtgagtgcngcctgccctccacgccccccctccagcgaagagatcagatgccttcatcggcagcaatcctcttggggtca

SEQ ID NO: 37 - DNA, 143 bp, Bos taurus
  misc_feature (72)..(72): n is a, c, g, or t, as described in Table 4
actcgggggcctgtcaggtgtccaggaggcaagtgaccactctgcgatccgggcgttccctcgggtgtcccntgcgcgcggatgtcctcctccaggacaggtctgccgcctgccgacgtccttccccgacgagcctttaaacc

SEQ ID NO: 38 - DNA, 143 bp, Bos taurus
  misc_feature (72)..(72): n is a, c, g, or t, as described in Table 4
gtggataatcggggtcactctgatttccccttcccagaaactgaggacggcgagccgtgtgtgttccccttngtgttcaacgggaagagctacgaggagtgtgttgtggagagcagggccaggctctggtgcgcgaccaccgc

SEQ ID NO: 39 - DNA, 143 bp, Bos taurus
  misc_feature (72)..(72): n is a, c, g, or t, as described in Table 4
tcgccytcccgcgcccactgtgtggagatcctcctgcccaggtcctggcaacccccatgctcctagtgttgnccagcgctggtcccgctatcttttctgagaaacacttggcttgttttagtgataggaagtcttggaacttg

SEQ ID NO: 40 - DNA, 143 bp, Bos taurus
  misc_feature (72)..(72): n is a, c, g, or t, as described in Table 4
cacgtggtggggggtggtggttgatccacctttagtgcgtttctcgttcagcctaaatcctcgcggtgactnctggtcagtggtcaggagctcagttgttgtagatgccaacccagaggtgtgtttcccgtctgatagaccga

SEQ ID NO: 41 - DNA, 143 bp, Bos taurus
  misc_feature (72)..(72): n is a, c, g, or t, as described in Table 4
aggagggtgagctctcaggcaagtgcacggcccrtggacagagctccgccttcctgctcgtgccccccacanccccagcgctggccttgccccccccctggagacccagtcacagcgcggggcggtgtctccgcaggtgaccc

SEQ ID NO: 42 - DNA, 143 bp, Bos taurus
  misc_feature (72)..(72): n is a, c, g, or t, as described in Table 4
gtccaccgagcactacctcatcaacgtgtgcaagtccctgtccccgcaggctggctcaggtgagcggggggngcgggggctyggggctcgtagggagtttgtgggggagaaagggagtcaggacggattcttcgaagtcatgt

SEQ ID NO: 43 - DNA, 143 bp, Bos taurus
  misc_feature (72)..(72): n is a, c, g, or t, as described in Table 4
ctccctcacggacgaacagctgtactacagcttcaacctgtccagcctctccaagagcaccttcaaggtaangccgtgccccagagcccgtgacctcggggcccctgccacctggcgtcactctcaggctcctctgtgtgtta

SEQ ID NO: 44 - DNA, 143 bp, Bos taurus
  misc_feature (72)..(72): n is a, c, g, or t, as described in Table 4
ctacgacctgcggctgctctcgtccctcaccggctcctggtccttcgtccacaacggagcctcgtgagtacntccccctaccagcctgccggctgtgtccgtcgccccgacgggrcgagtgtgcggcgcttcaagctccratt

SEQ ID NO: 45 - DNA, 143 bp, Bos taurus
  misc_feature (72)..(72): n is a, c, g, or t, as described in Table 4
ggccccccgatagtgagtgtggggcccgagggcagaggtcgccccgcarggggtttcagcggcccccccagngtgtcgtgtgtcttgctgtcggtggagagtcttcaggcagaacgaatggggacgtgagctgggactctgtg

SEQ ID NO: 46 - DNA, 143 bp, Bos taurus
  misc_feature (72)..(72): n is a, c, g, or t, as described in Table 4
cctggacaagcgcacgtgcacgcttttcttctcctggcacacacccctggcctgcgagcagacggtgagtcnggggcggcccagcccacccaacctaggggccttccacttctcccatgggtcctggggcaccccacccactt

SEQ ID NO: 47 - DNA, 143 bp, Bos taurus
  misc_feature (72)..(72): n is a, c, g, or t, as described in Table 4
gcctcgtaaaggcgggggaagagaagaaagcgggtgaaggcagggctcgtgactttctggagtgaggaaagngagggaggttctgctgtaggtgacacagaaactggggcggtccccggggagaagctgtcacgttgtctggc

SEQ ID NO: 48 - DNA, 143 bp, Bos taurus
  misc_feature (72)..(72): n is a, c, g, or t, as described in Table 4
ccccggtctcctctggccatgcaggtagtgacgtgggctgggtgcgagtcaccaggctggcrgtgctgaccnccagggagatgagtctccagagccacttctgaccttgaggctcctaggatgccctagagataacgtcagtt

SEQ ID NO: 49 - DNA, 143 bp, Bos taurus
  misc_feature (72)..(72): n is a, c, g, or t, as described in Table 4
tgtcactgcagaggtgtgggggcagtcccaagtatgcagagaggtctgtgtcttgtaaggctagagaggagncgcggtgggccagcctctgggggcgtttgataggcagtgcaccttcccttcttatttctctaaccacttag

SEQ ID NO: 50 - DNA, 143 bp, Bos taurus
  misc_feature (72)..(72): n is a, c, g, or t, as described in Table 4
tctggcctgggggccctctgatctctgggcctggagccctgagcttgttggcctgcacggctgccccagggntcccgtcctgtgtgccgactggcggtcctggcgtctctcacgtcatgggcactctctccctttctacctga

SEQ ID NO: 51 - DNA, 143 bp, Bos taurus
  misc_feature (72)..(72): n is a, c, g, or t, as described in Table 4
gtgccagacaggcgtgggcgatgctctgtccctgccatgtcctccgggactgggtttgaatgtgcctcttcncccctttcattcccgcaggtctgctccatcaaggaccccaacagcgggtacgtgtttgatctgaacccact

SEQ ID NO: 52 - DNA, 143 bp, Bos taurus
  misc_feature (72)..(72): n is a, c, g, or t, as described in Table 4
gggagtggccagggcatcgcgctctgcagggggggacaaggagggtgagctctcaggcaagtgcacggcccntggacagagctccgccttcctgctcgtgccccccacayccccagcgctggccttgccccccccctggagac

SEQ ID NO: 53 - DNA, 143 bp, Bos taurus
  misc_feature (72)..(72): n is a, c, g, or t, as described in Table 4
ccgtggccgccggcggaggcgcggtcgccaggccgagcagcctcagcgaggtcgggttgcgagctcggccgngcycggccgcgagcgccgagggcggcaggcgaggcccggccggcctggcacgcggcctggtcgggcggact

SEQ ID NO: 54 - DNA, 143 bp, Bos taurus
  misc_feature (72)..(72): n is a, c, g, or t, as described in Table 4
cgacagagaccacgagtggggcttctgcaagcactgtaagtggacacgccggggcccccgctggccggcgcngtagccctgcgcctggaggttctcttcctggactgtccacgttagtggcagcgcctctggtgcatgtggtg

SEQ ID NO: 55 - DNA, 143 bp, Bos taurus
  misc_feature (72)..(72): n is a, c, g, or t, as described in Table 4
cgtctgctgtcatcgagctgacctgtgccaagacagtggggcggccttcgttcacgaggtgagggtgcgggntaccccaccccagggggtagctgggcgctgggcgggctgggcccccgtcagaactcctccccgcggtttct

SEQ ID NO: 56 - DNA, 143 bp, Bos taurus
  misc_feature (72)..(72): n is a, c, g, or t, as described in Table 4
ggtyttgggcatcgggaagacgtttctggtaagacttgcggggtgcactctgatttgccttggaaggatggnaggaggcacagagctcaccctcatctcgtgacaggggaggcaggtgtccttaggagcctcccagggcacgg

SEQ ID NO: 57 - DNA, 143 bp, Bos taurus
  misc_feature (72)..(72): n is a, c, g, or t, as described in Table 4
ccgtgtgcaaggttcccgtggacggccccccgatagtgagtgtggggcccgagggcagaggtcgccccgcanggggtttcagcggcccccccagcgtgtcgtgtgtcttgctgtcggtggagagtcttcaggcagaacgaatg

SEQ ID NO: 58 - DNA, 143 bp, Bos taurus
  misc_feature (72)..(72): n is a, c, g, or t, as described in Table 4
ttcgtccacaacggagcctcgtgagtacytccccctaccagcctgccggctgtgtccgtcgccccgacgggncgagtgtgcggcgcttcaagctccrattctgaaggtggcacagcctcaggcctctgctcgggcaggcctgg

SEQ ID NO: 59 - DNA, 143 bp, Bos taurus
  misc_feature (72)..(72): n is a, c, g, or t, as described in Table 4
tacytccccctaccagcctgccggctgtgtccgtcgccccgacgggrcgagtgtgcggcgcttcaagctccnattctgaaggtggcacagcctcaggcctctgctcgggcaggcctggtgcattccagggggtttggaagcag

SEQ ID NO: 60 - DNA, 143 bp, Bos taurus
  misc_feature (72)..(72): n is a, c, g, or t, as described in Table 4
ccctgcctcagggcgggcgctgcaggtcagacgggaggacgctgtggctgtcccaggcctgtgcgcttcgcnaagccccttctcgtgtgtccccctttccttagcctcagactccttgttctacacctcggaggcggacgagt

SEQ ID NO: 61 - DNA, 143 bp, Bos taurus
  misc_feature (72)..(72): n is a, c, g, or t, as described in Table 4
ctgctgtgcataatgaaacacccactgtgtcagtagtgaagaacacagttggtctctccagagggaaagctnacagccacgtgtgttcgcagggctcgtactctgagaccgtctccatcagcaacctgggggtggcgaagacg

SEQ ID NO: 62 - DNA, 143 bp, Bos taurus
  misc_feature (72)..(72): n is a, c, g, or t, as described in Table 4
agtgcctttctggccgtgaaacccaccgtagccttttcaaggtcattgtattgtggttgtggtcccgcttcncacacactggtggttcattcggccaggaattgtgggctctgactcgggcgttgggtgaacacggcaggaag

SEQ ID NO: 63 - DNA, 143 bp, Bos taurus
  misc_feature (72)..(72): n is a, c, g, or t, as described in Table 4
gaggaaaaaatgtcccacctctttctaaatgctggctttgggtaacgagccccttctctgccgtccttcccntgtgtgtgtgtgtgtgtgtgtgtgtgtgtgtgtgtgtgtcccgagattaggaggaagataactctacatac

SEQ ID NO: 64 - DNA, 143 bp, Bos taurus
  misc_feature (72)..(72): n is a, c, g, t, or a deletion, as described in Table 4
gatgagtgcggtggtggccagaagataataacaaatataacactcatgtgcaaaccaggtacaaatgaaacncaaaatcagaaagcgcggggtctcccgggctcctgccaggggcgcccgagcattctctgtttgctgcgttt

SEQ ID NO: 65 - DNA, 143 bp, Bos taurus
  misc_feature (72)..(72): n is a, c, g, t, or a deletion, as described in Table 4
tttagagaggaagtggtcgtgcagccttgtgggctgaaacgcacttggccagctgggctgtgtttgttttgntttgttagatggtttatgattttgttccttgtcctcccgacagcttttctaagaacttaagtttacatggt

SEQ ID NO: 66 - DNA, 144 bp, Bos taurus
  misc_feature (72)..(73): n is a, c, g, or t, as described in Table 4
cacggcccrtggacagagctccgccttcctgctcgtgccccccacayccccagcgctggccttgcccccccnntggagacccagtcacagcgcggggcggtgtctccgcaggtgacccgaggcccgcacacctacagtgtgggg

SEQ ID NO: 67 - DNA, 143 bp, Bos taurus
  misc_feature (72)..(72): n is a, c, g, or t, as described in Table 4
tccatcaaggaccccaacagcgggtacgtgtttgatctgaacccactgaacaattcccgaggatacgtggtnttgggcatcgggaagacgtttctggtaagacttgcggggtgcactctgatttgccttggaaggatggragg

SEQ ID NO: 68 - DNA, 143 bp, Bos taurus
  misc_feature (72)..(72): n is a, c, g, or t, as described in Table 4
tggtgacggaagacagcaagttgaacctaggcgtcgtgcagatcagtcctcaggtgggcgccaacgggtccntgagcctcgtctacgtcaacggggacaagtgcaagaaccagcgtttctccaccaggataaacctcgagtgt

SEQ ID NO: 69 - DNA, 143 bp, Bos taurus
  misc_feature (72)..(72): n is a, c, g, or t, as described in Table 4
tcttcggcaggtctgtttaatcagaagctgacctacgagaatggggtgctgaagatgaactacaccgggggngacacctgccacaaggtgtaccagcgttccaccaccatctttttctactgcgaccgcagcacgcaggcggt

SEQ ID NO: 70 - DNA, 143 bp, Bos taurus
  misc_feature (72)..(72): n is a, c, g, or t, as described in Table 4
ccactcatccaccgcaccgggggttacgaagcatacgatgagagtgaggacgacggctccgacaccagcccngacttctacatcaacatctgccagccgctcaaccccatgcacgggttggcctgccccgccggcacggccgt

SEQ ID NO: 71 - DNA, 143 bp, Bos taurus
  misc_feature (72)..(72): n is a, c, g, or t, as described in Table 4
attcagcgcctctggggacgtgagaaccaacggggacaggtacatctacgagatccagctgtcgtccatcangggctccagcagccccgcctgctctggggccagcatctgccagaggaaggccaacgaccagcacttcagtc

SEQ ID NO: 72 - DNA, 143 bp, Bos taurus
  misc_feature (72)..(72): n is a, c, g, or t, as described in Table 4
atgaagggagctgggccttgaagccgcggcgctgacggtggatccgggtctggcgtgggggtggggtcgccntcccgcgcccactgtgtggagatcctcctgcccaggtcctggcaacccccatgctcctagtgttgmccagc

SEQ ID NO: 73 - DNA, 143 bp, Bos taurus
  misc_feature (72)..(72): n is a, c, g, or t, as described in Table 4
tggccgccggcggaggcgcggtcgccaggccgagcagcctcagcgaggtcgggttgcgagctcggccgrgcncggccgcgagcgccgagggcggcaggcgaggcccggccggcctggcacgcggcctggtcgggcggactctg

SEQ ID NO: 74 - DNA, 143 bp, Bos taurus
  misc_feature (72)..(72): n is a, c, g, or t, as described in Table 4
cttcaactacacctcactgatcacgttccactgtaagcggggcgtgagcatggtaagtgggcaccggtgtangaggcaccggtgtgcgggccggccagccagagccggaggccctcgaagcctgcctcggacgaaggctgccy

SEQ ID NO: 75 - DNA, 143 bp, Bos taurus
  misc_feature (72)..(72): n is a, c, g, or t, as described in Table 4
cactacctcatcaacgtgtgcaagtccctgtccccgcaggctggctcaggtgagcggggggygcgggggctnggggctcgtagggagtttgtgggggagaaagggagtcaggacggattcttcgaagtcatgtcactctctga

SEQ ID NO: 76 - DNA, 143 bp, Bos taurus
  misc_feature (72)..(72): n is a, c, g, or t, as described in Table 4
ygaggcaccggtgtgcgggccggccagccagagccggaggccctcgaagcctgcctcggacgaaggctgccngtgtccacagcgcctgcctcgcaccgtgtgctgtcagtggtgtgtggaatcactgcaggccctcagtttag

SEQ ID NO: 77 - DNA, 143 bp, Bos taurus
  misc_feature (72)..(72): n is a, c, g, or t, as described in Table 4
ggaattgtgggctctgactcgggcgttgggtgaacacggcaggaaggggtgagtgaggtggtggtggagaangcccgtccccagggcaaggtcggtggcgtctccatgccgtcgggccagcccagcctctcctgcaccccacg

SEQ ID NO: 78 - DNA, 143 bp, Bos taurus
  misc_feature (72)..(72): n is a, c, g, or t, as described in Table 4
ggcagattccactcaagtcaaagtggccgggagaccccagaacctgaccctccggtgggtatggcccccgcntgactctcaagggtgtcctgcatgtccctgtgaagcctaacacactcccctgccagatgcctgcttccatt

SEQ ID NO: 79 - DNA, 143 bp, Bos taurus
  misc_feature (72)..(72): n is a, c, g, or t, as described in Table 4
ggtctggttggagctcaggcagcctggaggggctgggatccggaaggacccttggctcctacaggtatggcnagttggaagtctagaacgggagctgtggtttgagatgctgccttgcttgggcaagactggggagttcaggc

SEQ ID NO: 80 - DNA, 143 bp, Bos taurus
  misc_feature (72)..(72): n is a, c, g, or t, as described in Table 4
ggttcctggagcagggggacggtgggagttgaggtcagggtctcagaagcctgagagccaagagtgctgtgngcctgactcagcatgattgtctatttattttgatgccctatttatattaacttattggtgcttcaaatggc

SEQ ID NO: 81 - DNA, 143 bp, Bos taurus
  misc_feature (72)..(72): n is a, c, g, or t, as described in Table 4
atcatcgcgtacctgggcgcctccctgggcaacatcacgagagaccagaaggtcctcaacccctacgcccanggcctgcacagcaagctgagcaccacggccgacgtcctgcggggtctgctcagcaacgtgctctgccgctt

SEQ ID NO: 82 - DNA, 143 bp, Bos taurus
  misc_feature (72)..(72): n is a, c, g, or t, as described in Table 4
ccccttcttctgggagactacagccgggcacgcagtgtcgggctggagtttggcccctgactcatcccctcngccagggtctttgtgagcaaaccccgaaagttgtctctggcgaccctgaccacggggtgagacagcagggg

SEQ ID NO: 83 - DNA, 143 bp, Bos taurus
  misc_feature (72)..(72): n is a, c, g, or t, as described in Table 4
gcaaaccccgaaagttgtctctggcgaccctgaccacggggtgagacagcaggggtcgggggcactaacccncgaccccccagcagaatgaccaccatcagtgccttggctgaccttgaaaggtctggttggagctcaggcag

SEQ ID NO: 84 - DNA, 143 bp, Bos taurus
  misc_feature (72)..(72): n is a, c, g, or t, as described in Table 4
gcgcccgggggactttcccagcgaggatgccctgtggaggctcagcaggcaggacttcctgcagaccctcancaccacactgggcctcatccttcgcatgctgagtgccctgcagcaggacctcccggaagcagcccaccaac

SEQ ID NO: 85 - DNA, 143 bp, Bos taurus
  misc_feature (72)..(72): n is a, c, g, or t, as described in Table 4
gggggagagacagggccgcagccgcagacacagcccctgccgggccctgaagaggggggcccgcaggacacngcccttcccggagatcaggagactcgcgcccaggggccagccgccccggtagcctttggggtgcccctgcc

SEQ ID NO: 86 - DNA, 143 bp, Bos taurus
  misc_feature (72)..(72): n is a, c, g, or t, as described in Table 4
agccagcctgggtgcagaccgagcgggagcagttccgggacttccgggatctgaacaaggacgggaagctgnacgggagtgaggtgggccactgggtgctgccccccgcccaggaccagcccctggtggaggccaaccactta

SEQ ID NO: 87 - DNA, 143 bp, Bos taurus
  misc_feature (72)..(72): n is a, c, g, or t, as described in Table 4
tgagggatcacccctttctcactggcagagtctcccagcccagaccaaggccccccgacatcaggctcagcntccaaaggcctccactaaccccccagctccaaatctgagcttcatcccacacaacggagaaacacacccct

SEQ ID NO: 88 - DNA, 143 bp, Bos taurus
  misc_feature (72)..(72): n is a, c, g, or t, as described in Table 4
cttggcctctcgaccaccagggacgtctctatggctcagaatattatctacagctcttgagaggaacttaangaccaaactcctattattttgtcctgtttgactgctttcctctgtttctgcattttctcagccccgatgcc

SEQ ID NO: 89 - DNA, 143 bp, Bos taurus
  misc_feature (72)..(72): n is a, c, g, or t, as described in Table 4
gctgacctggggcgccccagtggccaggcccccacctgtccagccctgcaggaggtggacaccgacctcagngtccccctgcccctgggcgctccacggactcaccactgggtcaacttctttccctgagacactgcagcccc

SEQ ID NO: 90 - DNA, 143 bp, Bos taurus
  misc_feature (72)..(72): n is a, c, g, or t, as described in Table 4
ttccctcacatgcgggacattgtgattgctgtgagtggggcctgaggaatccggcttcttacctcccttccngggacctaggcttctgacgccaagacttgcgtcccagcgtttaccttgggggccccagtgcccaccctcca

SEQ ID NO: 91 - DNA, 143 bp, Bos taurus
  misc_feature (72)..(72): n is a, c, g, or t, as described in Table 4
tgaaaggcccgggatccggaaatgtcagaaccaggctgggaggtcccgggaaccgcccctgatgtcaccccntctcgcccccgactcccccatcccagctgacctgtacacggcggagcccggggaggaggagccagcctggg

SEQ ID NO: 92 - DNA, 143 bp, Bos taurus
  misc_feature (72)..(72): n is a, c, g, or t, as described in Table 4
tttatgccattggagctgttctcagtatgtttcttattccataacggtgcttgtgttctacaaaattgattncagtttgagattgcatttgtttcgagtgcattttgtgaagttaggttttctttctaagattatcattgctg

SEQ ID NO: 93 - DNA, 143 bp, Bos taurus
  misc_feature (72)..(72): n is a, c, g, or t, as described in Table 4
tcattttgcattgtcctgttataatatagattgataattgtcataatagtagttcctartactttttaaacnatttcttgttttttttttttctttttctgtcgtttcagagatatacctagaatacctgacagcacacacac

SEQ ID NO: 94 - DNA, 143 bp, Bos taurus
  misc_feature (72)..(72): n is a, c, g, or t, as described in Table 4
catcctggaatccaaacctgaaccacaggtggagcttgttgtttcaaggcctattgggtgagttaatctgantactttcagttcagttcagttccgttcagtcgctcagtcgtgtctgactctttgcgacctcgtgaattgca

SEQ ID NO: 95 - DNA, 143 bp, Bos taurus
  misc_feature (72)..(72): n is a, c, g, or t, as described in Table 4
gtttattaagatatttaatggatcttgctatttcagtcgaaatgtggagcagggacttcgagggacacgctntgctataggacattataatacaattagccgaatggatagacaccgtgtcatggatgaccattattctccag

SEQ ID NO: 96 - DNA, 143 bp, Bos taurus
  misc_feature (72)..(72): n is a, c, g, or t, as described in Table 4
catatatttcaagaggtttgatggaaaggatttccacaagtcactggcaatatcaccaagtatttattgatntaaaaggaagttattaataccaggcaataaaagagcttaccatctcccaaaatactgatgatatgtatggt

SEQ ID NO: 97 - DNA, 143 bp, Bos taurus
  misc_feature (72)..(72): n is a, c, g, or t, as described in Table 4
aagatggaagtgtacctcgagattcaggagcaatgcttggcttgaaggtatgtgatgaaatatgtgagatgntctatattccttatagatttatcagaaaagcaaaagatataataactctataccaacttagtgtttttttt

SEQ ID NO: 98 - DNA, 143 bp, Bos taurus
  misc_feature (72)..(72): n is a, c, g, or t, as described in Table 4
cccaccccgtctgtgagcaggagctccttcctcctgtcctctgcagatggcagtttgtacgtctgtcacatntgcggtcttgactctgcttctttcctatttggcctcctagtgggcttccataagcaaagctcctagtcaga

SEQ ID NO: 99 - DNA, 143 bp, Bos taurus
  misc_feature (72)..(72): n is a, c, g, or t, as described in Table 4
acagttgacagctcatcttcctctggagagttcataatcagatatccagacagcactagttgataacaaccnacttctacctctctccaaatcagcctttgaaaaatgcttagattgaacagaggtttatgaggctgaactca

SEQ ID NO: 100 - DNA, 143 bp, Bos taurus
  misc_feature (72)..(72): n is a, c, g, or t, as described in Table 4
tagttcttaactgatttttaaaaggaaaataagcttacttcaaagcacaaaaacatcttaaatttaactagnttgacctctgaaatataatacaggctgtttcatgatttcattttctaataaataaaatgattaatttaaaa

SEQ ID NO: 101 - DNA, 143 bp, Bos taurus
  misc_feature (72)..(72): n is a, c, g, t, or a deletion, as described in Table 4
aggtaagcactatattctaatcatacatttgcctgtaattaataagagtttttcttttaggctgtatttctnagtagtcccttaataggtattacaacctttgtttttaagttctttaatggtgctaattatgtgaataaaat

SEQ ID NO: 102 - DNA, 144 bp, Bos taurus
  misc_feature (72)..(73): n is a, c, g, t, or a deletion, as described in Table 4
cagttcagttgccttgcttcattgttcttcttttacatttttgacgaagtccaagtctggagtaatctcttnnttgacagatggttttgatctaaattatccaagttctctctaattacctactgaagaaaaaaatgactgaac

SEQ ID NO: 103 - DNA, 143 bp, Bos taurus
  misc_feature (72)..(72): n is a, c, g, or t, as described in Table 4
tgttataagtgaaggactgacgtcctgggaagcatcaggtgaaaagcaagagaccaaagacgaggtctaggncagaacgtcagccctcccccggactagacaggagcagccggtccaaagtgacgacgtgagcagtggcagac

SEQ ID NO: 104 - DNA, 143 bp, Bos taurus
  misc_feature (72)..(72): n is a, c, g, or t, as described in Table 4
aggaagatcccctggaggaagaaatggcaacccattccagtatttktgcttggaaaatcccatggacagagnagcctggagggctacagtccatgcaagtcacaaagagttggacaggaatgaagcaattagcaggcacacac

SEQ ID NO: 105 - DNA, 143 bp, Bos taurus
  misc_feature (72)..(72): n is a, c, g, or t, as described in Table 4
tacacagttttgttcattttgcattgtcctgttataatatagattgataattgtcataatagtagttcctantactttttaaacratttcttgttttttttttttctttttctgtcgtttcagagatatacctagaatacctg

SEQ ID NO: 106 - DNA, 143 bp, Bos taurus
  misc_feature (72)..(72): n is a, c, g, or t, as described in Table 4
agtcctctccccagctgaaattcttggggaacttccaagcagtggccagkgctataaagctgtacacactanggaactatgctgaatgtaataaaccataatggaaaaaaaatatgaaaaaaagccaacacagtttcctttaa

SEQ ID NO: 107 - DNA, 143 bp, Bos taurus
  misc_feature (72)..(72): n is a, c, g, or t, as described in Table 4
ccttattatagcaatgtaattattatgtgcatgttaataaactatcaaattagatcataaaaatttcaaganatttgtcaaagtaaaatatctgaattaaactctccattcattgaagttattatagcatatccttttaagtt

SEQ ID NO: 108 - DNA, 143 bp, Bos taurus
  misc_feature (72)..(72): n is a, c, g, or t, as described in Table 4
agagatccggcttccatctctggctcaggaagatcccctggaggaagaaatggcaacccattccagtatttntgcttggaaaatcccatggacagagragcctggagggctacagtccatgcaagtcacaaagagttggacag

SEQ ID NO: 109 - DNA, 143 bp, Bos taurus
  misc_feature (72)..(72): n is a, c, g, or t, as described in Table 4
agtaaaatatctgaattaaactctccattcattgaagttattatagcatatccttttaagttaactgcaatntactaagtgaagtttatattctgtgctaatatcaggataagagaatgggccaaaggttgggaatgtaagca

SEQ ID NO: 110 - DNA, 143 bp, Bos taurus
  misc_feature (72)..(72): n is a, c, g, or t, as described in Table 4
ttattgatgaaatattatccttgtaaaaagtagaaaataaagcatatataaacaatttaattgtattgggcnggggtcatctctgtgatgattctaaaaatgtaattcaccagaaattgcttttgaatcattacatggaaaag

SEQ ID NO: 111 - DNA, 143 bp, Bos taurus
  misc_feature (72)..(72): n is a, c, g, or t, as described in Table 4
atggagtcaaacattctcttccagtcctctccccagctgaaattcttggggaacttccaagcagtggccagngctataaagctgtacacactarggaactatgctgaatgtaataaaccataatggaaaaaaaatatgaaaaa

SEQ ID NO: 112 - DNA, 143 bp, Bos taurus
  misc_feature (72)..(72): n is a, c, g, or t, as described in Table 4
tacttataaagtttaacataggcataatgattcctaggaatcaaaacagtgacagtaaatgctttgctattntttttaaacccatttcaaatgtttataatatagatgattttattctatatcaattttatattgtgtggatt

SEQ ID NO: 113 - DNA, 143 bp, Bos taurus
  misc_feature (72)..(72): n is a, c, g, or t, as described in Table 4
gattggccattgtcacctgtgagtagtgttggctggcctctggccctggttgacagttggttacaatcctgnctgtgttgccttccctcagagggatgcagcttatagactgggcagttctggttggtggctcctgcttctgg

SEQ ID NO: 114 - DNA, 143 bp, Bos taurus
  misc_feature (72)..(72): n is a, c, g, or t, as described in Table 4
gaggaagggtgggccagtaggagaggcctgaagtttaatgtctcttaattttcttaattagaatgcatttcntctcttgsaaaaatattacatcataaagtttttgttcaacataatcttctttaaattttaagggggctcaa

SEQ ID NO: 115 - DNA, 143 bp, Bos taurus
  misc_feature (72)..(72): n is a, c, g, or t, as described in Table 4
tatgtgaactatattcagaaatacatgaaatacgatgcaaagtagaaattatgygtatttaccaragatccngggatgatgagtttcatcaagttagaggtgtaaaccagcctctttgacaattagaacctttgtaaacttat

SEQ ID NO: 116 - DNA, 143 bp, Bos taurus
  misc_feature (72)..(72): n is a, c, g, or t, as described in Table 4
ttgcctggaaaatcccatggactgaggaacccagcaggctacatccatgaggatgcgtagagtctgacacgnctgaagtgacttagyacgcacgcatgcaggcatcaatgcggagtgggtcgggggagrcctgtctsctctta

SEQ ID NO: 117 - DNA, 143 bp, Bos taurus
  misc_feature (72)..(72): n is a, c, g, or t, as described in Table 4
ctgttttgttccaaagccctgttagctggcagacccacttaagcccatacaccagcactcaaaaatcagggntgccaaaaatgatgaaagctcagccttgatggggcttcccaggtgactcttgtaaagaacctacctgccaa

SEQ ID NO: 118 - DNA, 143 bp, Bos taurus
  misc_feature (72)..(72): n is a, c, g, or t, as described in Table 4
tacttttgtgagttattctcttagattctgtgtcttaaggtggcttttagtttattaagctgaagatactcntagagtgttcttcctgatgtaccatcattggaaggatgkatattttggtttaggtgaggcttttatgtttg

SEQ ID NO: 119 - DNA, 143 bp, Bos taurus
  misc_feature (72)..(72): n is a, c, g, or t, as described in Table 4
tgaaagaactacagttagagtagtggttttgcaactgacttgatcaatagccttagtaaagtccaggctggntttcagactaggatctagaattttttctcawtttgaggtactgtgatttataatgttaggaataactgact

SEQ ID NO: 120 - DNA, 143 bp, Bos taurus
  misc_feature (72)..(72): n is a, c, g, or t, as described in Table 4
aagagacttaagagacccaatccctgggttgggaagatcccctggaggagggcatggcaacccactccagtnttcttggcgggagaatcacatggacagaggaggctgcaggctgcagtccacagggtcacaaagagtcggac

SEQ ID NO: 121 - DNA, 143 bp, Bos taurus
  misc_feature (72)..(72): n is a, c, g, or t, as described in Table 4
tatagaaatgcacaagcaggtaagctattatttctttataagtgttttaaatgacagtaactgtgcactttngaaaggaagttgtatgttttgcagtttgattctgcacgtttttgtggccacctgtattttaaaagtccatg

SEQ ID NO: 122 - DNA, 143 bp, Bos taurus
  misc_feature (72)..(72): n is a, c, g, or t, as described in Table 4
aaactagaaatgtgccaggctatggaggaaagtattctgagattaaagttttgctgcagaaaatctacacantggacctttgtatgtgcagatggttgagaattaactttaccckatctaaacacatatttaaatataaaggg

SEQ ID NO: 123 - DNA, 143 bp, Bos taurus
  misc_feature (72)..(72): n is a, c, g, or t, as described in Table 4
gtcactgcctccttctaatgggcttgctgatgatagtttgcctcttccttaggaaatactcctgccagaatntaaagtgtgttttaatatcagcctgctaatatttcgggaatttgtaaccagctgactgttctctttattgc

SEQ ID NO: 124 - DNA, 143 bp, Bos taurus
  misc_feature (72)..(72): n is a, c, g, or t, as described in Table 4
rcctgtctsctcttaacagcttggtgagcgtatactaagagcaaaaaggagataaagtctcatgtgattttnaaaaatgacagggttaaatgactggtcatctctcaattctgctttcctttctaattccagagctcttcggt

SEQ ID NO: 125 - DNA, 143 bp, Bos taurus
  misc_feature (72)..(72): n is a, c, g, or t, as described in Table 4
ccctcagagggatgcagcttatagactgggcagttctggttggtggctcctgcttctggtgtccctgggccnagcaccctgtcttcctctttgttgccctcagcttctgcaatccttttgcatgacgtatgcagggtctactg

SEQ ID NO: 126 - DNA, 143 bp, Bos taurus
  misc_feature (72)..(72): n is a, c, g, or t, as described in Table 4
ggggtacgtggcctttccattttagctctgatcatcttagtgtttgtcactggctctctctcgctctctctntaaattttgttcaattgaagaggcaaaaggcagtagaggatcacacagtgaaatggagcactttgccttca

SEQ ID NO: 127 - DNA, 143 bp, Bos taurus
  misc_feature (72)..(72): n is a, c, g, or t, as described in Table 4
cagcctcgactgagaatgtgacatgtgacctttttattttttagagaacgtgacttttatatgttttagagncaaaaccactttctactcctgatagttgaaattggagaccaaacgaggagaactttacagggtcctgtcag

SEQ ID NO: 128 - DNA, 143 bp, Bos taurus
  misc_feature (72)..(72): n is a, c, g, or t, as described in Table 4
gtgggccagtaggagaggcctgaagtttaatgtctcttaattttcttaattagaatgcatttcmtctcttgnaaaaatattacatcataaagtttttgttcaacataatcttctttaaattttaagggggctcaatatttatt

SEQ ID NO: 129 - DNA, 143 bp, Bos taurus
  misc_feature (72)..(72): n is a, c, g, or t, as described in Table 4
cttttattaccaaagcagttaggatttccatataataggattcatatattttattattttttttattttcantttgtttctgtcctctttagcttttattagacattaccttctttttttcaatataccaatatgtgtttact

SEQ ID NO: 130 - DNA, 143 bp, Bos taurus
  misc_feature (72)..(72): n is a, c, g, or t, as described in Table 4
gagaataagcttctgttttcagccacctggtttgtgggagtttgcttggcagccctagtaaactaatatagntcccaagagttaagtttatctgtcagttttgtttcatcatcaggatagttagtaattgccatgtgatacta

SEQ ID NO: 131 - DNA, 143 bp, Bos taurus
  misc_feature (72)..(72): n is a, c, g, or t, as described in Table 4
gctttgatgatctgattcagaattatgctactttaacattcatgtaaggtttctgttgtacacttagttgtnttcatttttaattaccaagagtggaagtaggcaacataatctttctcctcttaagtgcttttaaaagtctt

SEQ ID NO: 132 - DNA, 143 bp, Bos taurus
  misc_feature (72)..(72): n is a, c, g, or t, as described in Table 4
cacatggcaacccackgcagtattcttgcctggagaatcccatggacagaggagcctggtgggctacagccngtggggttgcaaagacttagatacgtccaagtgactgacactttcctcactttcacggtctttctttgcgt

SEQ ID NO: 133 - DNA, 143 bp, Bos taurus
  misc_feature (72)..(72): n is a, c, g, or t, as described in Table 4
atagaaattagtaggaattatgtgaactatattcagaaatacatgaaatacgatgcaaagtagaaattatgngtatttaccaragatccmgggatgatgagtttcatcaagttagaggtgtaaaccagcctctttgacaatta

SEQ ID NO: 134 - DNA, 144 bp, Bos taurus
  misc_feature (72)..(73): n is a, c, g, t, or a deletion, as described in Table 4
gcgatacctccaaacctgacaggcattccaggaggaaagccgtgagtaccaagctctgtgcctcgtgttcanngtgtgtctgggccctcactggccctttagactctgagaactactgggcagtgttggcaagttccttcagct

SEQ ID NO: 135 - DNA, 143 bp, Bos taurus
  misc_feature (72)..(72): n is a, c, g, t, or a deletion, as described in Table 4
aggtacagtccctgtgctcaggctccaggaatatagtggtgaaaaggacagaaatgtttactgtcctggggntcaacgttttatttttattggggaagagacacattcattgcaggattaacaatgatgaaattgcttcagtg

SEQ ID NO: 136 - DNA, 144 bp, Bos taurus
  misc_feature (72)..(73): n is a, c, g, t, or a deletion, as described in Table 4
ttttactaaccattcacatttaagatagtttgtcctctccaaattggccgctgctttcacagtgtgactctnngttcttaacaaatttgctagtatatttacatgatccaactgtaaggaaaaaagatctgtgtttaatgtttc

SEQ ID NO: 137 - DNA, 143 bp, Bos taurus
  misc_feature (72)..(72): n is a, c, g, or t, as described in Table 4
ctatcacatggacatggtctgccagtgcctctgaccccacacccttcggggcttcagcctcctgccattccncccattggtagcagtgccggccttctggccctctccagtgccctgggaggccagtcccaccttccaattaa

SEQ ID NO: 138 - DNA, 143 bp, Bos taurus
  misc_feature (72)..(72): n is a, c, g, or t, as described in Table 4
agcattaaacataaataacttctagtatgcttatttctaattctttgttttgctggctttagtttttttttnactgtgccactccttatatatattaagacttatagttttattcaagggagattgttgttaaaaagtcacgt

SEQ ID NO: 139 - DNA, 143 bp, Bos taurus
  misc_feature (72)..(72): n is a, c, g, or t, as described in Table 4
aatactgatttatttgcagctccttctcttcaggctgagtgcacagcagtgtcatgaggtgagagtcggtcngtcttgggcttggcagggtgcgtctgagggaacaaggacacttgcatcatctggatgcagggggtacacag

SEQ ID NO: 140 - DNA, 143 bp, Bos taurus
  misc_feature (72)..(72): n is a, c, g, or t, as described in Table 4
tagagtctgacacgrctgaagtgacttagyacgcacgcatgcaggcatcaatgcggagtgggtcgggggagncctgtctsctcttaacagcttggtgagcgtatactaagagcaaaaaggagataaagtctcatgtgattttw

SEQ ID NO: 141 - DNA, 143 bp, Bos taurus
  misc_feature (72)..(72): n is a, c, g, or t, as described in Table 4
acattttggtttctcttacttttgtatctagaaagtatctcatatataactttcccctaagaaaaattaaanttctagtataacttaaatttggcttattgtcagacactgaaaccacaggctcagaatacagttasagtgat

SEQ ID NO: 142 - DNA, 143 bp, Bos taurus
  misc_feature (72)..(72): n is a, c, g, or t, as described in Table 4
attaaarttctagtataacttaaatttggcttattgtcagacactgaaaccacaggctcagaatacagttanagtgattggccattgtcacctgtgagtagtgttggctggcctctggccctggttgacagttggttacaatc

SEQ ID NO: 143 - DNA, 143 bp, Bos taurus
  misc_feature (72)..(72): n is a, c, g, or t, as described in Table 4
gaaaatatttatactccagtgcacacttttgcgtcagtttcattttatagttcctcacgccagagtagggtntattttgaaatcgtatataatcattcaagatgagtctgggagtaagtatctgtgtagcttggaaaccaggg

SEQ ID NO: 144 - DNA, 143 bp, Bos taurus
  misc_feature (72)..(72): n is a, c, g, or t, as described in Table 4
taggaattatgtgaactatattcagaaatacatgaaatacgatgcaaagtagaaattatgygtatttaccanagatccmgggatgatgagtttcatcaagttagaggtgtaaaccagcctctttgacaattagaacctttgta

SEQ ID NO: 145 - DNA, 143 bp, Bos taurus
  misc_feature (72)..(72): n is a, c, g, or t, as described in Table 4
aagtttttgttcaacataatcttctttaaattttaagggggctcaatatttatttgtttaaactggaatttnaattttagaagcatttgtttctcaaaatgtagataacccaggcagttggggttttaacactcacttccctt

SEQ ID NO: 146 - DNA, 143 bp, Bos taurus
  misc_feature (72)..(72): n is a, c, g, or t, as described in Table 4
ttaccaaccaaaactagaccacaagataacattctaggagagaaaactagttaatacagttgtagttgagtntcagttggctgactgaaagcctgtgtttgcaggtgagtgagccaggaaacagtgtttgatctggcaaccga

SEQ ID NO: 147 - DNA, 143 bp, Bos taurus
  misc_feature (72)..(72): n is a, c, g, or t, as described in Table 4
gtttttttagtccgaatcaagcacctagcacttaccctgtctgacacatagtaggtgttcagtaaattaagncaaatgtttgaaccttgatgaaagcttaaatgacttttgcaaacattaaaataagcttatttgaattacag

SEQ ID NO: 148 - DNA, 143 bp, Bos taurus
  misc_feature (72)..(72): n is a, c, g, or t, as described in Table 4
gacacgrctgaagtgacttagyacgcacgcatgcaggcatcaatgcggagtgggtcgggggagrcctgtctnctcttaacagcttggtgagcgtatactaagagcaaaaaggagataaagtctcatgtgattttwaaaaatga

SEQ ID NO: 149 - DNA, 143 bp, Bos taurus
  misc_feature (72)..(72): n is a, c, g, or t, as described in Table 4
gccagccttcctatggggggcccatattctgaatgtctctgtgtacttcccaatggtgtcacgaagactttntgctgcaktgcaccaagaagagtctttcttatgatgagggaataggtagaagaatgacatctaggtttgca

SEQ ID NO: 150 - DNA, 143 bp, Bos taurus
  misc_feature (72)..(72): n is a, c, g, or t, as described in Table 4
attaatatagtgatcttttaaatgggtgtaggccttttttttttctttctggtggaattgattgagcagtwnaacatgaatcttcccagaatggaccccyatgagatactttttaatgtttctaaacagaaagttgaggtggt

SEQ ID NO: 151 - DNA, 143 bp, Bos taurus
  misc_feature (72)..(72): n is a, c, g, or t, as described in Table 4
accagatggtagcctggcatttttgttatggaggtttctgttcttgagaacaccttgcataatttcagtgcntacatactcccattcctcatcactgtaccagaactgcaacagcctcttgatctgactctttggcagagaat

SEQ ID NO: 152 - DNA, 143 bp, Bos taurus
  misc_feature (72)..(72): n is a, c, g, or t, as described in Table 4
tcctatggggggcccatattctgaatgtctctgtgtacttcccaatggtgtcacgaagactttstgctgcantgcaccaagaagagtctttcttatgatgagggaataggtagaagaatgacatctaggtttgcatgtatgtt

SEQ ID NO: 153 - DNA, 149 bp, Bos taurus
  misc_feature (72)..(78): n is a, c, g, t, or a deletion, as described in Table 4
tttctgaaattatgtcaaaggtagcttggtgctctgtggatctggtcaagtagtaattaattttaattaatnnnnnnnacagaaaagttgacatctgtgttatttattatttagtagagatcaaatttgacaagtgtgtgattttatgt

SEQ ID NO: 154 - DNA, 143 bp, Bos taurus
  misc_feature (72)..(72): n is a, c, g, or t, as described in Table 4
ctttcctggaagttaacgaaaatatctaaaaggcagcttagtatagagtgaaaacatgcacttgtagccacngtcatgggttctaggcaggtctactgcctgctctctttgtgatcttggacaataataataaaaagtaatta

SEQ ID NO: 155 - DNA, 143 bp, Bos taurus
  misc_feature (72)..(72): n is a, c, g, or t, as described in Table 4
gccggccttctggccctctccagtgccctgggaggccagtcccaccttccaattaaagatgagaagaagcancatgacagtgatcaccaaagaggtgagtgattttctcagaatgtctgtctggtatcacctgtctgctgctg

SEQ ID NO: 156 - DNA, 143 bp, Bos taurus
  misc_feature (72)..(72): n is a, c, g, or t, as described in Table 4
taaaataaatatgataaattttgtagtatttttattgacctcgatactgaatattttctacagcaatttgangagtcttaacagtctgttccagaacattttttgctcctaagctattgaagacttctggcttgaaacgtcca

SEQ ID NO: 157 - DNA, 143 bp, Bos taurus
  misc_feature (72)..(72): n is a, c, g, or t, as described in Table 4
cattaatatagtgatcttttaaatgggtgtaggccttttttttttctttctggtggaattgattgagcagtnkaacatgaatcttcccagaatggaccccyatgagatactttttaatgtttctaaacagaaagttgaggtgg

SEQ ID NO: 158 - DNA, 143 bp, Bos taurus
  misc_feature (72)..(72): n is a, c, g, or t, as described in Table 4
caactgacttgatcaatagccttagtaaagtccaggctggrtttcagactaggatctagaattttttctcantttgaggtactgtgatttataatgttaggaataactgactttaaagcttctcttttattaccaaagcagtt

SEQ ID NO: 159 - DNA, 143 bp, Bos taurus
  misc_feature (72)..(72): n is a, c, g, or t, as described in Table 4
gaggatgagatggctggatggcatcaccgactcaatggacatgagtttgaatgaactcygggagttggtganggacagggaggcctggtgtgctgcggttcatggcgtcgcaaagagttggacacgactgagtgactggacta

SEQ ID NO: 160 - DNA, 143 bp, Bos taurus
  misc_feature (72)..(72): n is a, c, g, or t, as described in Table 4
catggactgaggaacccagcaggctacatccatgaggatgcgtagagtctgacacgrctgaagtgacttagnacgcacgcatgcaggcatcaatgcggagtgggtcgggggagrcctgtctsctcttaacagcttggtgagcg

SEQ ID NO: 161 - DNA, 143 bp, Bos taurus
  misc_feature (72)..(72): n is a, c, g, or t, as described in Table 4
tatacatgaattggcagtaagtgattttagaaatgtttgtttacctttggaatatattacatgatttttaanatgttgtttccttttcagattattttctgtagaagtccataagaagtatttgcttttgtgggaggagtcca

SEQ ID NO: 162 - DNA, 143 bp, Bos taurus
  misc_feature (72)..(72): n is a, c, g, or t, as described in Table 4
tggacatttttttggtcaccataatgccctcgatcacttgataattccttgatagcttctagcttctaatanctagcctacaaacagatttctatgattatttcaaataattggtttgcaagagtttccctccttttaaaatt

SEQ ID NO: 163 - DNA, 143 bp, Bos taurus
  misc_feature (72)..(72): n is a, c, g, or t, as described in Table 4
taggccttttttttttctttctggtggaattgattgagcagtwkaacatgaatcttcccagaatggaccccnatgagatactttttaatgtttctaaacagaaagttgaggtggtggtaggcggggctgaaggctgtgcataa

SEQ ID NO: 164 - DNA, 143 bp, Bos taurus
  misc_feature (72)..(72): n is a, c, g, or t, as described in Table 4
gaaggggatgacagaggatgagatggctggatggcatcaccgactcaatggacatgagtttgaatgaactcngggagttggtgawggacagggaggcctggtgtgctgcggttcatggcgtcgcaaagagttggacacgactg

SEQ ID NO: 165 - DNA, 143 bp, Bos taurus
  misc_feature (72)..(72): n is a, c, g, or t, as described in Table 4
gatcattttatttggtgaggagaaacagaatggtgtgtattctggggctttaataggaaggatccaaggcanctgcttgtcacttggccatccagtacccacgttcatgtgcccattgtaagccctggatttagaggctgaac

SEQ ID NO: 166 - DNA, 143 bp, Bos taurus
  misc_feature (72)..(72): n is a, c, g, or t, as described in Table 4
atactttttaatgtttctaaacagaaagttgaggtggtggtaggcggggctgaaggctgtgcataacgatgntctttataatactcagaaggttaaatgtggataaacactgaaaacaaggcttcagaaaagcctcagtatta

SEQ ID NO: 167 - DNA, 143 bp, Bos taurus
  misc_feature (72)..(72): n is a, c, g, or t, as described in Table 4
gtggtcaggtgcttctcaaaagtggtaatgagtgtggattcagcaatgtcagtaggtagggggtgggcctgngatgctgcatttcttacaagctctcagaagatctcatggctgctggacagtgaaccataccttgagtaacg

SEQ ID NO: 168 - DNA, 143 bp, Bos taurus
  misc_feature (72)..(72): n is a, c, g, or t, as described in Table 4
aacttgcctgctgtgcctaggaattagagtccatagagtaccacattttcatcagacctttgtgagtcatcngcttgtgatgtacaaagatccttggaggtgttaagaatgctatgtttgagcttgattttcttacttttgtg

SEQ ID NO: 169 - DNA, 143 bp, Bos taurus
  misc_feature (72)..(72): n is a, c, g, or t, as described in Table 4
taaagttttgctgcagaaaatctacacartggacctttgtatgtgcagatggttgagaattaactttacccnatctaaacacatatttaaatataaagggaatttcgttattgcagatagttcagcctcgactgagaatgtga

SEQ ID NO: 170 - DNA, 143 bp, Bos taurus
  misc_feature (72)..(72): n is a, c, g, or t, as described in Table 4
gtggcttttagtttattaagctgaagatactcrtagagtgttcttcctgatgtaccatcattggaaggatgnatattttggtttaggtgaggcttttatgtttgcttggggacattttgaacaaactaggaagcttgtttgat

SEQ ID NO: 171 - DNA, 143 bp, Bos taurus
  misc_feature (72)..(72): n is a, c, g, or t, as described in Table 4
tgcaggagacataagagacatgggtttgatccctcggtctggaagagtcccaggagcacatggcaacccacngcagtattcttgcctggagaatcccatggacagaggagcctggtgggctacagccygtggggttgcaaaga

SEQ ID NO: 172 - DNA, 41 bp, Sus scrofa
tcttacacatcaggagatagytccgaggtggatttctacaa

SEQ ID NO: 173 - DNA, 41 bp, Sus Scrofa; identical to SEQ ID NO: 172

SEQ ID NO: 174 - DNA, 41 bp, Sus Scrofa; identical to SEQ ID NO: 172

SEQ ID NO: 175 - DNA, 41 bp, Sus Scrofa
tcttacacatcaggagatggytccgaggtggatttctacaa

(19) United States
(12) Patent Application Publication
(10) Pub. No.: US 2011/0123983 A1
(43) Pub. Date: May 26, 2011

(54) Methods of Using Genetic Markers and Related Epistatic Interactions

(75) Inventors: Fengxing Du, St. Louis, MO (US); Edward J. Cargill, St. Louis, MO (US); Michael D. Lohuis, St. Louis, MO (US); Michael D. Grosz, St. Louis, MO (US)

(73) Assignee: Pfizer Inc.

(21) Appl. No.: 12/674,164

(22) PCT Filed: Sep. 8, 2008
(86) PCT No.: PCT/US08/10480

§ 371 Date: Aug. 20, 2010

Related U.S. Application Data
(60) Provisional application No. 60/971,750, filed on Sep. 12, 2007.

(51) Int. Cl. C12Q 1/68

(52) U.S. Cl. 435/6

(57) ABSTRACT

The present invention provides methods for improving desirable animal traits including improved fitness and productivity in dairy animals. Also provided are methods for determining a dairy animal's genotype with respect to multiple markers associated with fitness and/or productivity. The invention also provides methods for selecting or allocating animals for predetermined uses such as progeny testing or nucleus herd breeding, for picking potential parent animals for breeding, and for producing improved progeny animals. Each of the above methods may be further improved through the incorporation of interaction effects between multiple SNPs.

### PRIORITY CLAIM

This application claims the benefit of U.S. Provisional Application Ser. No. 60/971,750 filed Sep. 12, 2007, which is herein incorporated by reference in its entirety.

### INCORPORATION OF SEQUENCE LISTING

A sequence listing containing the file named pa_CandGeneInteractionEffects2_annotated.ST25.txt, which is 84,218 bytes (as measured in Microsoft Windows®) was created on Sep. 5, 2008, comprises 175 nucleotide sequences, is submitted herewith, and is herein incorporated by reference in its entirety.

### FIELD OF THE INVENTION

The present invention relates to the enhancement of desirable characteristics in dairy cattle. More specifically, it relates to the use of genes and genetic markers in methods for improving dairy cattle with respect to fitness and/or productivity traits using genetic markers, including simultaneous application of multiple genetic markers and interactions between specific alleles at those markers.

### BACKGROUND OF THE INVENTION

The future viability and competitiveness of the dairy industry depends on continual improvement in milk productivity (e.g. milk, fat, protein yield, fat %, protein % and persistency of lactation), health (e.g. Somatic Cell Count, mastitis incidence), fertility (e.g. pregnancy rate, display of estrus, calving interval and non-return rates in bulls), calving ease (e.g. direct and maternal calving ease), longevity (e.g. productive life), and functional conformation (e.g. udder support, proper foot and leg shape, proper rump angle, etc.). Unfortunately efficiency traits are often unfavorably correlated with fitness traits. Although fitness traits all have some degree of underlying genetic variation in commercial cattle populations, the accuracy of selecting breeding animals with superior genetic merit for many of them is low due to low heritability or the inability to measure the trait cost effectively on the candidate animal. In addition, many productivity and fitness traits can only be measured on females. Thus, the accuracy of conventional selection for these traits is moderate to low and ability to make genetic change through selection is limited, particularly for fitness traits.

In addition, there are frequently interactions between specific alleles at multiple loci which confound prediction of genetic merit. In other words, the effects of combinations of alleles on traits may not be strictly additive, but rather synergistic (or antagonistic). In the absence of an understanding of these interactions, a priori estimation of genetic merit is obviously more difficult and less accurate.

Genomics offers the potential for greater improvement in productivity and fitness traits through the discovery of genes, or genetic markers linked to genes, that account for genetic variation and can be used for more direct and accurate selection. Close to 1000 markers with associations with productivity and fitness traits have been reported (see www.bovineqtl.tamu.edu/ for a searchable database of reported QTL), however, the resolution of QTL location is still quite low which makes it difficult to utilize these QTL in marker-assisted selection (MAS) on an industry scale. Only a few QTL have been fully characterized with a strong putative or well-confirmed causal mutation: DGAT1 on chromosome 14 (Grisard et al., 2002; Winter et al, 2002; Kuhn et al., 2004) GHR on chromosome 20 (Blott et al., 2003), ABCG2 (Cohen-Zinder et al., 2005) or SPP1 on chromosome 6 (Schnabel et al., 2005). However, these discoveries are rare and only explain a small portion of the genetic variance for productivity traits and no genes controlling fitness traits have been fully characterized. A more successful strategy employs the use of whole-genome high-density scans of the entire bovine genome in which QTL are mapped with sufficient resolution to explain the majority of genetic variation around productivity and fitness traits.

Cattle herds used for milk production around the world originate predominantly from the Holstein or Holstein-Friesian breeds which are known for high levels of production. However, the high production levels in Holsteins have also been linked to greater calving difficulty and reduced levels of fertility. It is unclear whether these unfavorable correlations are due to pleiotropic gene effects or simply due to linked genes. If the latter is true, with marker knowledge, it may be possible to select for favorable recombinants that contain the favorable alleles from several linked genes that are normally at frequencies too low to allow much progress with traditional selection. Since Holstein germplasm has been sold and transported globally for several decades, the Holstein breed has effectively become one large global population held to relatively moderate inbreeding rates. Also, the outbred nature of such a large population selected for several generations has allowed linkage disequilibrium to break down except within relatively short distances (i.e. less than a few centimorgans) (Hayes et al., 2006). In addition, as a result of selection in several countries with different breeding goals, linkage disequilibrium between relatively close loci can be quite variable due to the effects of drift within sub-populations that have become mixed via several generations of global selection and breeding. Given this pattern of linkage disequilibrium, very dense marker coverage is required to refine QTL locations with sufficient precision to find markers that are in very tight linkage disequilibrium with them. Therefore, markers that are in very tight linkage disequilibrium with the QTL are essential for effective population-wide MAS or whole-genome selection (WGS).

Most productivity and fitness traits are quantitative in nature and hence are governed by hundreds of QTL of small to moderately sized effects. Therefore, to characterize enough QTL to explain a majority of variation for these traits, the whole genome must be scanned with a set of markers mapped to the genome at high resolution (i.e. greater than 1 marker/cM); otherwise known as whole-genome analysis.

Furthermore, a sufficient number of QTL must be used in MAS in order to accurately predict the breeding value of an animal without phenotyping records on relatives or the animal itself. The application of such a high-density whole-genome marker map to discover and finely-map QTL explaining variation in productivity and fitness traits is described herein. The large number of resulting linked markers can be used in several methods of marker selection or marker-assisted selection, including whole-genome selection (WGS) (Meuwissen et al., Genetics 2001) to improve the genetic merit of the population for these traits and create value in the dairy industry.

Unlike some simple traits which may be fully explained by one causal mutation, many productivity and fitness traits require a large number of markers to accurately predict the phenotypic performance of the animal Quantitative phenotypes generally involve multiple genes, multiple pathways, and complex interactions. In some cases, this complexity results in interactions between markers which is exceptionally difficult to predict.

Few studies have investigated the contribution of the interactions between candidate gene SNPs to quantitative variation in dairy traits. There are several possible reasons for lack of such interaction studies. First, different candidate genes were generally investigated by different groups, and genotypes of different candidate genes were collected on different animals; focuses of most candidate gene studies were to discover/confirm an association between a trait and SNP(s) of their interest; investigation of interaction effects generally needs larger sample sizes.

However, expression of quantitative traits are results of interactions of multiple physiological pathways (for example lipid metabolism, appetite/satiety, etc.), and a large number of genes are generally involved in each physiological pathway. Therefore, it appears to be reasonable to expect some degree of interaction among genes that are involved in the same or different pathways, and to expect a proportion of genetic quantitative variation are due to such interactions.

The inventors have identified markers associated with novel traits in important genes in dairy cows, as well as numerous interaction effects including epistatic effects between these genes, which can be used to substantially improve the accuracy of genetic evaluations, prediction, and selection.

### SUMMARY OF THE INVENTION

This section provides a non-exhaustive summary of the present invention.

Various embodiments of the invention provide methods for evaluating an animal's genotype at 1 or more positions in the animal's genome. In various aspects of these embodiments the animal's genotype is evaluated at positions within a segment of DNA (an allele) that contains at least two SNPs selected from the SNPs described in the Tables and Sequence Listing. For each of the SNPs listed in tables 1 and 3, details regarding SNP location, SNP length, and alleles can be found in Table 4.

Other embodiments of the invention provide methods for allocating animals for use according to their predicted marker breeding value for productivity and/or fitness traits. Various aspects of this embodiment of the invention provide methods that comprise: a) analyzing the animal's genomic sequence at two or more polymorphisms (where the alleles analyzed each comprise at least two SNP) to determine the animal's genotype at each of those polymorphisms; b) analyzing the genotype determined for each polymorphisms to determine which allele of the SNP is present; c) allocating the animal for use based on its genotype at two or more of the polymorphisms analyzed. Various aspects of this embodiment of the invention provide methods for allocating animals for use based on a favorable association between the animal's genotype, at two or more polymorphisms disclosed in the present application, and a desired phenotype. Alternatively, the methods provide for not allocating an animal for a certain use because it has two or more SNP alleles that are either associated with undesirable phenotypes or are not associated with desirable phenotypes.

Other embodiments of the invention provide methods for selecting animals for use in breeding to produce progeny. Various aspects of these methods comprise: A) determining the genotype of at least two potential parent animals at two or more locus/loci, where at least two of the loci analyzed contains an allele of a SNP selected from the group of SNPs described in Tables 1 and 3. B) Analyzing the determined genotype at two or more positions for at least two animals to determine which of the SNP alleles is present. C) Correlating the analyzed allele(s) with two or more phenotypes. D) Allocating at least two animals for use to produce progeny. Alternative embodiments include analyzing the animal's genotype at two or more loci wherein the analysis comprises evaluating interaction effects.

Other embodiments of the invention provide methods for producing offspring animals (progeny animals). Aspects of this embodiment of the invention provide methods that comprise: breeding an animal that has been selected for breeding by methods described herein to produce offspring. The offspring may be produced by purely natural methods or through the use of any appropriate technical means, including but not limited to: artificial insemination; embryo transfer (ET), multiple ovulation embryo transfer (MOET), in vitro fertilization (IVF), or any combination thereof.

Other embodiments of the invention provide for methods of selecting animals for use in breeding to produce progeny wherein interaction effects between multiple markers are applied in the analysis.

### DEFINITIONS

The following definitions are provided to aid those skilled in the art to more readily understand and appreciate the full scope of the present invention. Nevertheless, as indicated in the definitions provided below, the definitions provided are not intended to be exclusive, unless so indicated. Rather, they are preferred definitions, provided to focus the skilled artisan on various illustrative embodiments of the invention.

As used herein the term “allelic association” preferably means: nonrandom deviation of f(AᵢBⱼ) from the product of f(Aᵢ) and f(Bⱼ), which is specifically defined by r²>0.2, where r² is measured from a reasonably large animal sample (e.g., ≧100) and defined as

$${r}^{2} = \frac{{[ f ( {A}_{1} {B}_{1} ) - f ( {A}_{1} ) f ( {B}_{1} ) ]}^{2}}{f ( {A}_{1} ) ( 1 - f ( {A}_{1} ) ) ( f ( {B}_{1} ) ( 1 - f ( {B}_{1} ) )} \qquad [ Equation 1 ]$$

where A₁ represents an allele at one locus, B₁ represents an allele at another locus; f(A₁B₁) denotes frequency of having both A₁ and B₁, f(A₁) is the frequency of A₁, f(B₁) is the frequency of B₁ in a population.

As used herein the terms “allocating animals for use” and “allocation for use” preferably mean deciding how an animal will be used within a herd or that it will be removed from the herd to achieve desired herd management goals. For example, an animal might be allocated for use as a breeding animal or allocated for sale as a non-breeding animal (e.g. allocated to animals intended to be sold for meat). In certain aspects of the invention, animals may be allocated for use in sub-groups within the breeding programs that have very specific goals (e.g. productivity or fitness). Accordingly, even within the group of animals allocated for breeding purposes, there may be more specific allocation for use to achieve more specific and/or specialized breeding goals.

As used herein the terms “animal” or “animals” preferably refer to dairy cattle.

As used herein “fitness” preferably refers to traits that include, but are not limited to: pregnancy rate (PR), daughter pregnancy rate (DPR), productive life (PL), somatic cell count (SCC) and somatic cell score (SCS). PR and DPR refer to the percentage of non-pregnant animals that become pregnant during each 21-day period. PL is calculated as months in milk in each lactation, summed across all lactations until removal of the cow from the herd (by culling or death). SCS=log₂(SCC/100,000)+3, where SCC is somatic cells per milliliter of milk.

As used herein the term “growth” refers to the measurement of various parameters associated with an increase in an animal's size/weight.

As used herein the term “linkage disequilibrium” preferably means allelic association wherein A₁ and B₁ (as used in the above definition of allelic association) are present on the same chromosome.

As used herein the term “marker-assisted selection (MAS) preferably refers to the selection of animals on the basis of marker information in possible combination with pedigree and phenotypic data.

As used herein the terms “marker breeding value (MBV)” and “predicted marker breeding value (PMBV)” refer to an estimate of an animal's genetic transmitting ability with respect to specific traits and is based on its genotype.

As used herein the term “natural breeding” preferably refers to mating animals without human intervention in the fertilization process. That is, without the use of mechanical or technical methods such as artificial insemination or embryo transfer. The term does not refer to selection of the parent animals.

As used herein the term “net merit” preferably refers to a composite index that includes several commonly measured traits weighted according to relative economic value in a typical production setting and expressed as lifetime economic worth per cow relative to an industry base. Examples of a net merit indexes include, but are not limited to, $NM or TPI in the USA, LPI in Canada, etc (formulae for calculating these indices are well known in the art (e.g. $NM can be found on the USDA/AIPL website: www.aipl.arsusda.gov/reference.htm).

As used herein the term “predicted value” preferably refers to an estimate of an animal's breeding value or transmitting ability based on its genotype and pedigree.

As used herein “productivity” and “production” preferably refers to yield traits that include, but are not limited to: total milk yield, milk fat percentage, milk fat yield, milk protein percentage, milk protein yield, total lifetime production, milking speed and lactation persistency.

As used herein the term “quantitative trait” is used to denote a trait that is controlled by multiple (two or more, and often many) genes each of which contributes small to moderate effect on the trait. The observations on quantitative traits often follow a normal distribution.

As used herein the term “quantitative trait locus (QTL)” is used to describe a locus that contains polymorphism that has an effect on a quantitative trait.

As used herein the term “reproductive material” includes, but is not limited to semen, spermatozoa, ova, and zygote(s).

As used herein the term “single nucleotide polymorphism” or “SNP” refer to a location in an animal's genome that is polymorphic within the population. That is, within the population some individual animals have one type of base at that position, while others have a different base. For example, a SNP might refer to a location in the genome where some animals have a “G” in their DNA sequence, while others have a “T”.

As used herein the terms “hybridization under stringent conditions” and “stringent hybridization conditions” preferably mean conditions under which a “probe” will hybridize to its target sequence to a detectably greater degree than to other sequences (e.g., at least 5-fold over background). Stringent conditions are target-sequence-dependent and will differ depending on the structure of the polynucleotide. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing).

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringency may also be adjusted with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. The duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the thermal melting point (Tₘ) can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with 90% identity are sought, the Tₘ can be decreased 10° C. Generally, stringent conditions are selected to be about 5° C. lower than the Tₘ for the specific sequence and its complement at a defined ionic strength and pH.

However, highly stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See also Sambrook et al. (1989) Molecular Cloning: A Laboratory Manual (2d ed., Cold Spring Harbor Laboratory Press, Plainview, N.Y.).

As used herein the terms “marker breeding value (MBV)” and “predicted marker breeding value (PMBV)” respectively refer to an estimate of an animal's genetic transmitting ability with respect to either productivity traits or fitness traits that is based on its genotype.

As used herein the term “whole-genome analysis” preferably refers to the process of QTL mapping of the entire genome at high marker density (i.e. approximately one marker per cM) and detection of markers that are in population-wide linkage disequilibrium with QTL.

As used herein the term “whole-genome selection (WGS)” preferably refers to the process of marker-assisted selection (MAS) on a genome-wide basis in which markers spanning the entire genome at moderate to high density (e.g. approximately one marker per 1-5 cM), or at moderate to high density in QTL regions, or directly neighboring or flanking QTL that explain a significant portion of the genetic variation controlling two or more traits.

As used herein, the term “interaction effect” preferably refers to an alteration of the predicted phenotypic effect of a first marker, depending on the allelic state of a second marker. For example, if SNP1 has an effect estimate of 10 for a positive allelic association when SNP2 is an A, but SNP1 has an effect estimate of 5 for a positive allelic association when SPP2 is a T, the change in effect estimate from 10 to 5 would be considered an interaction effect. Marker-based interaction effects must involve at least two markers.

As used herein, the term “epistatic interaction” preferably refers to interactions between alleles of genes, for example when the action of one gene is modified by one or several genes that assort independently (but may be linked).

### ILLUSTRATIVE EMBODIMENTS OF THE INVENTION

Various embodiments of the present invention provide methods for evaluating an animal's (especially a dairy animal's) genotype at 1 or more positions in the animal's genome. Aspects of these embodiments of the invention provide methods that comprise determining the animal's genomic sequence at 1 or more locations (loci) that contain single nucleotide polymorphisms (SNPs). Specifically, the invention provides methods for evaluating an animal's genotype by determining which of two or more alleles for the SNP are present for each of 1 or more SNPs selected from the group consisting of the SNPs described in Tables 1 and 3 of the instant application.

In preferred aspects of these embodiments the animal's genotype is evaluated to determine which allele is present for 10 or more SNPs selected from the group of SNPs described in Tables 1 and 3. More, preferably the animal's genotype is determined for positions corresponding with 2, 10, 100, 200, 500, or 1000, or more of SNPs, at least two of which are described in Tables 1 and 3. In some embodiments of this invention, interactions between two SNPs are used in analysis of the animal's genotype.

In other aspects of this embodiment, the animal's genotype is analyzed with respect to at least 1 or more SNPs that have been shown to be associated with productivity and/or fitness (see Table 1 for a list of the SNPs associated with these traits). Further, embodiments of the invention provides a method for genotyping 2 or more, 10 or more, 10 or more, 50 or more, 100 or more, 200 or more, or 500 or more, or 1000 or more SNPs, at least one of which has been determined to be significantly associated with a productivity or fitness trait as described in Table 1.

Aspects of the present invention also provides for both whole-genome analysis and whole genome-selection (WGS) (that is marker-assisted selection (MAS) on a genome-wide basis). Various aspects of this embodiment of the invention provide for either whole-genome analysis or WGS wherein the makers analyzed for an animal span the animal's entire genome at moderate to high density. That is, the animal's genome is analyzed with markers that on average occur, at least, approximately every 1 to 5 centimorgans in the genome. Moreover the invention provides that of the markers used to carry out the whole-genome analysis or WGS, including 2 or more, 10 or more, 10 or more, 50 or more, 100 or more, 200 or more, 500 or more, or 1000 or more markers, at least one of which are selected from the markers described in Tables 1 and 3. In preferred aspects of this embodiment the markers may be associated with fitness or productivity traits, or may be associated with both fitness and productivity traits.

In any embodiment of the invention the genomic sequence at the SNP locus may be determined by any means compatible with the present invention. Suitable means are well known to those skilled in the art and include, but are not limited to direct sequencing, sequencing by synthesis, primer extension, Matrix Assisted Laser Desorption/Ionization-Time Of Flight (MALDI-TOF) mass spectrometry, polymerase chain reaction-restriction fragment length polymorphism, microarray/multiplex array systems (e.g. those available from Affymetrix, Santa Clara, Calif.), and allele-specific hybridization.

Other embodiments of the invention provide methods for allocating animals for subsequent use (e.g. to be used as sires or dams or to be sold for meat or dairy purposes) according to their predicted value for productivity or fitness. Various aspects of this embodiment of the invention comprise determining at least two animal's genotype for at least two SNPs selected from the group of SNPs described in Tables 1 and 3 (methods for determining animals' genotypes for two or more SNPs are described supra). Thus, the animal's allocation for use may be determined based on its genotype at one or more, 2 or more, 10 or more, 10 or more, 50 or more, 100 or more, 300 or more, or 500 or more, or 1000 or more SNPs. The animal's allocation may further include an analysis of interaction effects between at least two SNPs.

The instant invention provides embodiments where analysis of the genotypes of the SNPs described in Tables 1 and 3 is the only analysis done. Other embodiments provide methods where analysis of the SNPs disclosed herein is combined with any other desired type of genomic or phenotypic analysis (e.g. analysis of any genetic markers beyond those disclosed in the instant invention). Moreover, the SNPs analyzed may be selected from those SNPs only associated productivity, only associated with fitness, or the analysis may be done for SNPs selected from any desired combination of fitness and productivity. SNPs associated with various traits are listed in Table 1.

According to various aspects of these embodiments of the invention, once the animal's genetic sequence for the selected SNP(s) have been determined, this information is evaluated to determine which allele of the SNP is present for at least two of the selected SNPs. Preferably the animal's allelic complement for all of the determined SNPs is evaluated. Finally, the animal is allocated for use based on its genotype for two or more of the SNP positions evaluated. Preferably, the allocation is made taking into account the animal's genotype at each of the SNPs evaluated, but its allocation may be based on any subset or subsets of the SNPs evaluated.

According to various aspects of embodiments of the invention, once the animal's genetic sequence for the selected SNP(s) have been determined, this information is evaluated to determine which allele of the SNP is present for at least two of the selected SNPs. Preferably the animal's allelic complement for all of the determined SNPs is evaluated. An analysis of the allelic orientations of the SNPs is performed, and preferably, the result of the analysis includes information related to at least one interaction effect. Finally, the animal is allocated for use based on its genotype for two or more of the SNP positions evaluated. Preferably, the allocation is made taking into account the animal's genotype at each of the SNPs evaluated, but its allocation may be based on any subset or subsets of the SNPs evaluated.

The allocation may be made based on any suitable criteria. For any SNP, a determination may be made as to whether one of the allele(s) is associated/correlated with desirable characteristics or associated with undesirable characteristics. Furthermore, this determination may preferably include information related to interaction effects between multiple makers. This determination will often depend on breeding or herd management goals. Determination of which alleles are associated with desirable phenotypic characteristics can be made by any suitable means. Methods for determining these associations are well known in the art; moreover, aspects of the use of these methods are generally described in the EXAMPLES, below.

Phenotypic traits that may be associated with the SNPs of the current invention include, but are not limited to; fitness traits and productivity traits. Fitness traits include but are not limited to: pregnancy rate (PR), daughter pregnancy rate (DPR), productive life (PL), somatic cell count (SCC) and somatic cell score (SCS). Productivity traits include but are not limited to: total milk yield, milk fat percentage, milk fat yield, milk protein percentage, milk protein yield, total lifetime production, milking speed and lactation persistency

According to various aspects of this embodiment of the invention allocation for use of the animal may entail either positive selection for the animals having the desired genotype(s) (e.g. the animals with the desired genotypes are selected for productivity traits), negative selection of animals having undesirable genotypes (e.g. animals with an undesirable genotypes are culled from the herd), or any combination of these methods. According to preferred aspects of this embodiment of the invention animals identified as having SNP alleles associated with desirable phenotypes are allocated for use consistent with that phenotype (e.g. allocated for breeding based on phenotypes positively associated with fitness). Alternatively, animals that do not have SNP genotypes that are positively correlated with the desired phenotype (or possess SNP alleles that are negatively correlated with that phenotype) are not allocated for the same use as those with a positive correlation for the trait.

Other embodiments of the invention provide methods for selecting potential parent animals (i.e., allocation for breeding) to improve fitness and/or productivity in potential offspring. Various aspects of this embodiment of the invention comprise determining at least two animal's genotype for at least two SNPs selected from the group of SNPs described in Tables 1 and 3. Furthermore, determination of whether and how an animal will be used as a potential parent animal may be based on its genotype at two or more, 2 or more, 10 or more, 50 or more, 100 or more, 300 or more, or 500 or more, including at least one of the SNPs described in Tables 1 and 3. Moreover, as with other types of allocation for use, various aspects of these embodiments of the invention provide methods where the only analysis done is to genotype the animal for two or more of the SNPs described in Tables 1 and 3. Other aspects of these embodiments provide methods where analysis of two or more SNPs disclosed herein is combined with any other desired genomic or phenotypic analysis (e.g. analysis of any genetic markers beyond those disclosed in the instant invention). Moreover, the SNP(s) analyzed may all be selected from those associated only with fitness traits or only with productivity traits. Conversely, the analysis may be done for SNPs selected from any desired combination of these or other traits.

According to various aspects of these embodiments of the invention, once the animal's genetic sequence at the site of the selected SNP(s) have been determined, this information is evaluated to determine which allele of the SNP is present for at least two of the selected SNPs. Preferably the animal's allelic complement for all of the sequenced SNPs is evaluated. Additionally, the animal's allelic complement is analyzed and correlated with the probability that the animal's progeny will express two or more phenotypic traits. Finally, the animal is allocated for breeding use based on its genotype for two or more of the SNP positions evaluated and the probability that it will pass the desired genotype(s)/allele(s) to its progeny. Preferably, the breeding allocation is made taking into account the animal's genotype at each of the SNPs evaluated. However, its breeding allocation may be based on any subset or subsets of the SNPs evaluated.

The breeding allocation may be made based on any suitable criteria. For example, breeding allocation may be made so as to increase the probability of enhancing a single certain desirable characteristic in a population, in preference to other characteristics, (e.g. increased fitness, or even specifically lowering somatic cell score (SCS) as part of fitness); alternatively, the selection may be made so as to generally maximize overall production based on a combination of traits. The allocations chosen are dependent on the breeding goals. Sub-categories falling within fitness, include, inter alia: daughter pregnancy rate (DPR), productive life (PL), and somatic cell score. Sub-categories falling within productivity include, inter alia: milk fat percentage, milk fat yield, total milk yield, milk protein percentage, and total milk protein.

Other embodiments of the instant invention provide methods for producing progeny animals. According to various aspects of this embodiment of the invention, the animals used to produce the progeny are those that have been allocated for breeding according to any of the embodiments of the current invention. Those using the animals to produce progeny may perform the necessary analysis or, alternatively, those producing the progeny may obtain animals that have been analyzed by another. The progeny may be produced by any appropriate means, including, but not limited to using: (i) natural breeding, (ii) artificial insemination, (iii) in vitro fertilization (IVF) or (iv) collecting semen/spermatozoa and/or at least two ovum from the animal and contacting it, respectively with ova/ovum or semen/spermatozoa from a second animal to produce a conceptus by any means.

According to preferred aspects of this embodiment of the invention the progeny are produced by a process comprising natural breeding. In other aspects of this embodiment the progeny are produced through a process comprising the use of standard artificial insemination (AI), in vitro fertilization, multiple ovulation embryo transfer (MOET), or any combination thereof.

Other embodiments of the invention provide for methods that comprise allocating an animal for breeding purposes and collecting/isolating genetic material from that animal: wherein genetic material includes but is not limited to: semen, spermatozoa, ovum, zygotes, blood, tissue, serum, DNA, and RNA.

It is understood that most efficient and effective use of the methods and information provided by the instant invention employ computer programs and/or electronically accessible databases that comprise all or a portion of the sequences disclosed in the instant application. Accordingly, the various embodiments of the instant invention provide for databases comprising all or a portion of the sequences corresponding to at least 2 SNPs described in Tables 1 and 3. In preferred aspect of these embodiments the databases comprise sequences for 1 or more, 5 or more, 10 or more, 20 or more, 50 or more, or substantially all of the SNPs described in Tables 1 and 3.

It is further understood that efficient analysis and use of the methods and information provided by the instant invention will employ the use of automated genotyping; particularly when large numbers (e.g. 100s) of markers are evaluated. Any suitable method known in the art may be used to perform such genotyping, including, but not limited to the use of micro-arrays.

Other embodiments of the invention provide methods wherein two or more of the SNP sequence databases described herein are accessed by two or more computer-executable programs. Such methods include, but are not limited to, use of the databases by programs to analyze for an association between the SNP and a phenotypic trait, or other user-defined trait (e.g. traits measured using two or more metrics such as gene expression levels, protein expression levels, or chemical profiles), and programs used to allocate animals for breeding or market.

Other embodiments of the invention provide methods comprising collecting genetic material from an animal that has been allocated for breeding. Wherein the animal has been allocated for breeding by any of the methods disclosed as part of the instant invention.

Other embodiments of the invention provide for diagnostic kits or other diagnostic devices for determining which allele of a SNP is present in a sample; wherein the SNP(s) are selected from the group of SNPs described in Tables 1 and 3. In various aspects of this embodiment of the invention, the kit or device provides reagents/instruments to facilitate a determination as to whether nucleic acid corresponding to the SNP is present. Such kit/or device may further facilitate a determination as to which allele of the SNP is present. In certain aspects of this embodiment of the invention the kit or device comprises at least two nucleic acid oligonucleotide suitable for DNA amplification (e.g. through polymerase chain reaction). In other aspects of the invention the kit or device comprises a purified nucleic acid fragment capable of specifically hybridizing, under stringent conditions, with at least two allele of at least two SNPs described in Tables 1 and 3.

In particularly preferred aspects of this embodiment of the invention the kit or device comprises at least two nucleic acid array (e.g. DNA micro-arrays) capable of determining which allele of two or more of the SNPs described in Tables 1 and 3 is present in a sample. Preferred aspects of this embodiment of the invention provide DNA micro-arrays capable of simultaneously determining which allele is present in a sample for 2 or more SNPs. Preferably, the DNA micro-array is capable of determining which SNP allele is present in a sample for 10 or more, 50 or more, 100 or more, 200 or more, 500 or more, or 1000 or more SNPs. Methods for making such arrays are known to those skilled in the art and such arrays are commercially available (e.g. from Affymetrix, Santa Clara, Calif.).

Genetic markers for fitness and/or productivity that are in allelic association with any of the SNPs described in the Tables may be identified by any suitable means known to those skilled in the art. For example, a genomic library may be screened using a probe specific for any of the sequences of the SNPs described in the Tables. In this way clones comprising at least a portion of that sequence can be identified and then up to 300 kilobases of 3′ and/or 5′ flanking chromosomal sequence can be determined. By this means, genetic markers in allelic association with the SNPs described in the Tables will be identified.

Other embodiments of the present invention provide methods for identifying genes that may be associated with phenotypic variation. According to various aspects of these embodiments, the chromosomal location of a SNP associated with a particular phenotypic variation can be determined, by means well known to those skilled in the art. Once the chromosomal location is determined genes suspected to be involved with determination of the phenotype can be analyzed. Such genes may be identified by sequencing adjacent portions of the chromosome or by comparison with analogous section of the human genetic map (or known genetic maps for other species). An early example of the existence of clusters of conserved genes is reviewed in Womack (1987), where genes mapping to the same chromosome in one species were observed to map to the same chromosome in other, closely related, species. As mapping resolution improved, reports of the conservation of gene structure and order within conserved chromosomal regions were published (for example, Grosz et al, 1992). More recently, large scale radiation hybrid mapping and BAC sequence have yielded chromosome-scale comparative mapping predictions between human and bovine genomes (Everts-van der Wind et al., 2005), between human and porcine genomes (Yasue et al., 2006) and among vertebrate genomes (Demars et al., 2006)

Other embodiments of the invention provide methods for identifying causal mutations that underlie two or more quantitative trait loci (QTL). Various aspects of this embodiment of the invention provide for the identification QTL that are in allelic association with two or more of the SNPs described in Tables 1 and 3. Once these SNPs are identified, it is within the ability of skilled artisans to identify mutations located proximal to such SNP(s). Further, one skilled in the art can identify genes located proximate to the identified SNP(s) and evaluate these genes to select those likely to contain the causal mutation. Once identified, these genes and the surrounding sequence can be analyzed for the presence of mutations, in order to identify the causal mutation.

Furthermore, once genes associated with phenotypic variation have been identified, the accuracy of the analysis can be improved by investigating interaction effects. In absence of interaction effects among QTL, one can utilize the marginal effect of individual QTL for faster genetic improvement. In general, one would estimate the breeding value of each allele or genotype and use the estimated breeding values in conjunction with animal's polygenic breeding value to make breeding decisions.

In presence of interaction effects, the true breeding value of a haplotype consisting of polymorphisms from multiple QTL is different from the summation of the breeding value of individual polymorphisms at each QTL. Therefore, the approach as described above designed for absence of nonallleic interaction is suboptimum. Instead, one should estimate breeding values of haplotypes or genotype configurations for optimization of genetic improvement.

The population frequencies of haplotypes are used in estimating breeding value of an haplotype. In presence of population-wise linkage disequilibrium, the frequency of a haplotype is different from the product of corresponding allelic frequencies. In this case, it is more appropriate to use haplotype frequencies for breeding value estimation.

Additional benefits from appropriately using interaction effects in a breeding program come from the difference between the true breeding value of a haplotype and the sum of the breeding values of the corresponding alleles. The sizes of differences are determined by the magnitude of interaction effects and the extent of population-wise linkage disequilibrium between interactive QTL.

Interaction effects can also be used to produce genetically superior crossbred or hybrid animals for higher efficiency of commercial production. As an example, assume that genotype A₁A₂B₁B₂ is the best genotype and is better than the summation of the breeding values (and genotypic values) of genotype A₁A₂ and B₁B₂. One way to utilize it is to create two lines with genotype A₁A₁B₁B₁ and A₂A₂B₂B₂ respectively. These two lines could be from different breeds to create an ideal crossbred, or from within an existing breed population to create an ideal hybrid. Crossbreeds or hybrids created from these two lines will all have genotypes A₁A₂B₁B₂, which improves the efficiency of commercial production.

Interaction effects can also be used within computer mating programs to produce genetically superior offspring for higher efficiency of commercial production. As an example, assume that genotype A₁A₂B₁B₂ is the best genotype and is better than the summation of the breeding values (and genotypic values) of genotype A₁A₂ and B₁B₂. One way to utilize it is to identify which cows and bulls have genotype A₁A₁B₁B₁ and A₂A₂B₂B₂. When a potential mates are ranked in the mating program, the interaction effects could also be included when calculating the estimated breeding value of potential offspring. For example, for individuals with the A₁A₁B₁B₁, potential mates with the A₂A₂B₂B₂ genotype would have additional mating value from the favorable interaction term. Of course, this idea could be extended for multiple interactions between multiple sets of loci with favorable or unfavorable interactions. Since managing this amount of information would be extremely difficult in the normal application of artificial insemination, computer mating could be used to manage and optimize matings.

As mentioned above (see paragraph 76), two distinct sub-lines could be created from within an existing breed population in order to create the ideal hybrid via crossing these two sub-lines, such that all commercial offspring have genotypes A₁A₂B₁B₂, which improves the efficiency of commercial production. However, the creation of sub-lines optimized for maximizing the interaction terms for multiple interactions could be quite complicated. One solution is the use of computer mating to create ideal sub-lines for eventual use in producing optimized hybrids. Depending on the ultimate commercial value of different traits, several sub-lines could be created to optimize the interaction effects for different breeding goals.

### EXAMPLES

The following examples are included to demonstrate general embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the invention.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied without departing from the concept and scope of the invention.

### Example 1

### Determining Associations Between Genetic Markers and Phenotypic Traits

Simultaneous discovery and fine-mapping on a genome-wide basis of genes underlying quantitative traits (Quantitative Trait Loci: QTL) requires genetic markers densely covering the entire genome. As described in this example, a whole-genome, dense-coverage marker map was constructed from microsatellite and single nucleotide polymorphism (SNP) markers with previous estimates of location in the bovine genome, and from SNP markers with putative locations in the bovine genome based on homology with human sequence and the human/cow comparative map. A new linkage-mapping software package was developed, as an extension of the CRIMAP software (Green et al., Washington University School of Medicine, St. Louis, 1990), to allow more efficient mapping of densely-spaced markers genome-wide in a pedigreed livestock population (Liu and Grosz Abstract C014; Grapes et al. Abstract W244; 2006 Proceedings of the XIV Plant and Animal Genome Conference, www.intl-pag.org). The new linkage mapping tools build on the basic mapping principles programmed in CRIMAP to improve efficiency through partitioning of large pedigrees, automation of chromosomal assignment and two-point linkage analysis, and merging of sub-maps into complete chromosomes. The resulting whole-genome discovery map (WGDM) included 6,966 markers and a map length of 3,290 cM for an average map density of 2.18 markers/cM. The average gap between markers was 0.47 cM and the largest gap was 7.8 cM. This map provided the basis for whole-genome analysis and fine-mapping of QTL contributing to variation in productivity and fitness in dairy cattle.

### Discovery and Mapping Populations

Systems for discovery and mapping populations can take many forms. The most effective strategies for determining population-wide marker/QTL associations include a large and genetically diverse sample of individuals with phenotypic measurements of interest collected in a design that allows accounting for non-genetic effects and includes information regarding the pedigree of the individuals measured. In the present example, an outbred population following the grand-daughter design (Weller et al., 1990) was used to discover and map QTL: the population, from the Holstein breed, had 529 sires each with an average of 6.1 genotyped sons, and each son has an average of 4216 daughters with milk data. DNA samples were collected from approximately 3,200 Holstein bulls and about 350 bulls from other dairy breeds; representing multiple sire and grandsire families.

### Phenotypic Analyses

Dairy traits under evaluation include traditional traits such as milk yield (“MILK”) (pounds), fat yield (“FAT”) (pounds), fat percentage (“FATPCT”) (percent), productive life (“PL”) (months), somatic cell score (“SCS”) (Log), daughter pregnancy rate (“DPR”) (percent), protein yield (“PROT”) (pounds), protein percentage (“PROTPCT”) (percent), and net merit (“NM”) (dollar). These traits are sex-limited, as no individual phenotypes can be measured on male animals. Instead, genetic merits of these traits defined as PTA (predicted transmitting ability) were estimated using phenotypes of all relatives. Most dairy bulls were progeny tested with a reasonably larger number of daughters (e.g., >50), and their PTA estimation is generally more or considerably more accurate than individual cow phenotype data. The genetic evaluation for traditional dairy traits of the US Holstein population is performed quarterly by USDA. Detailed descriptions of traits, genetic evaluation procedures, and genetic parameters used in the evaluation can be found at the USDA AIPL web site (www.aipl.arsusda.gov). It is meaningful to note that the dairy traits evaluated in this example are not independent: FAT and PROT are composite traits of MILK and FATPCT, and MILK and PROTPCT, respectively. NM is an index trait calculated based on protein yield, fat yield, production life, somatic cell score, daughter pregnancy, calving difficulty, and several type traits. Protein yield and fat yield together account for >50% of NM, and the value of milk yield, fat content, and protein content is accounted for via protein yield and fat yield.

PTA data of all bulls with progeny testing data were downloaded from the USDA evaluation published at the AIPL site in November 2005. The PTA data were analyzed using the following two models:

yᵢⱼ=sᵢ+PTAdᵢⱼ [Equation 2]

yᵢ=μ+β₁(SPTA)ᵢ+PTAd [Equation 3]

where yᵢ (yᵢⱼ) is the PTA of the i^{th} bull (PTA of the j^{th} son of the i^{th} sire); sᵢ is the effect of the i^{th} sire; (SPTA)ᵢ is the sire's PTA of the i^{th} bull of the whole sample; μ is the population mean; PTAdᵢ (PTAdᵢⱼ) is the residual bull PTA.

Equation 2 is referred to as the sire model, in which sires were fitted as fixed factors. Among all USA Holstein progeny tested bulls, a considerably large number of sires only have a very small number of progeny tested sons (e.g., some have one son), and it is clearly undesirable to fit sires as fixed factors in these cases. It is well known the USA Holstein herds have been making steady and rapid genetic progress in traditional dairy traits in the last several decades, implying that the sire's effect can be partially accounted for by fitting the birth year of a bull. For sires with <10 progeny tested sons, sires were replaced with son's birth year in Equation 2. Equation 3 is referred to as the SPTA model, in which sire's PTA are fitted as a covariate. Residual PTA (PTAdᵢ or PTAdᵢⱼ) were estimated using linear regression.

### SNP-Trait Association Analyses

In the present example, linkage disequilibrium (LD) mapping was performed in the aforementioned discovery population using statistical analyses based on probabilities of individual ordered genotypes estimated conditional on observed marker genotypes. The first step was to estimate sire's ordered genotype probabilities at all linked markers conditional on grandsire's and offspring marker genotype data. The exact calculation quickly becomes computationally infeasible as the size and complexity of the pedigree and number of linked markers increases. For example, there are, in total 2^{k} ordered genotypes for all linked loci when a sire has k linked heterozygous loci. A stepwise procedure developed based on a likelihood ratio test was used for estimating probabilities of sire's ordered genotypes at all linked markers.

The probabilities of ordered genotypes at loci of interest were estimated conditional on flanking informative markers as follows:

$$P ( {H}_{sik} {H}_{dlk} \vert M ) = {\sum }_{a}^{ } {\sum }_{b}^{ } P ( {H}_{sa} {H}_{db} \vert M ) * P ( {H}_{sik} {H}_{dlk} \vert {H}_{sa} {H}_{db} , M ) \qquad [ Equation 4 ]$$

Where P(HₛₐH_{db}|M) is the probability of sire having a pair of haplotypes (or order genotype) HₛₐH_{db} at all linked loci conditional on the observed genotype data M, and P(HₛᵢₖH_{dlk}|HₛₐH_{db},M) is the probability of a son having ordered genotype HₛᵢₖH_{dlk} at loci of interest conditional on sire's ordered genotype HₛₐH_{db} at all linked loci and the observed genotype data M.

To determine associations between haplotypes probabilities and trait phenotypes, haplotypes of neighboring (and/or non-neighboring) markers across each chromosome were defined by setting the maximum length of a chromosomal interval and minimum and maximum number of markers to be included. Clearly, one needs to set similar parameters to form or define groups of marker loci for haplotype evaluation. The association between pre-adjusted trait phenotypes and haplotype (or pair of haplotype that is alternatively termed as ordered genotypes) was evaluated via a regression approach with the following models:

$$\begin{matrix}{PTAd}_{k} = {\sum }_{i}^{ } {β}_{si} P ( {H}_{sik} ) + {e}_{k} & [ Equation 5 ] \\ {PTAd}_{k} = {\sum }_{i}^{ } {β}_{di} P ( {H}_{dik} ) + {e}_{k} & [ Equation 6 ] \\ {PTAd}_{k} = {\sum }_{i}^{ } {β}_{si} [ P ( {H}_{sik} ) + P ( {H}_{dik} ) ] + {e}_{k} & [ Equation 7 ] \\ {PTAd}_{k} = {\sum }_{i}^{ } {β}_{si} [ P ( {H}_{sik} {H}_{djk} ) + P ( {H}_{sjk} {H}_{dik} ) ] + {e}_{k} & [ Equation 8 ]\end{matrix}$$

where PTAdₖ is the preadjusted PTA of the k^{th} bull as defined in Equation 3 under the sire model and can be replaced with PTAdi as defined in Equation 3 under the SPTA model, and eₖ is the residual; P(Hₛᵢₖ) and P(H_{dik}) are the probability of paternal and maternal haplotype of individual k being haplotype i; P(HₛᵢₖH_{dik}) is the probability of individual k has paternal haplotype i and maternal haplotype j that can be estimated using Equation 4; all β are corresponding regression coefficients. Equations 5, 6, 7, and 8 are designed to model paternal haplotype, maternal haplotype, additive haplotype, and genotype effects, respectively.

Least-squares methods were used to estimate the effect of a haplotype or haplotype pair on a phenotypic trait and the regular F-test used to test the significance of the effect. Permutation tests were performed based on phenotype permutation (20,000) within each paternal half-sib family to estimate Type I error rate (p value).

### Example 2

### Analyzing for Interaction Effects Between Multiple Genetic Markers

Clustering of SNPs from a candidate gene. Mainly due to small effective population sizes and strong selection, alleles from tightly-linked SNPs are generally associated in animal populations (e.g., Farnir et al., 2000; Du et al., 2007). Clearly, if two SNPs are in perfect LD, their association with traits of interest and interaction with other SNPs on traits of interest will be similar, which doesn't provide much additional statistical evidence. It is, therefore, helpful to cluster SNPs from the same candidate gene when multiple SNPs at a single gene are genotyped.

Trait phenotype preadjustment. This study focuses on traditional dairy traits, including milk yield (“MILK”) (pounds), fat yield (“FAT”) (pounds), fat percentage (“FATPCT”) (percent), productive life (“PL”) (months), somatic cell score (“SCS”) (Log), daughter pregnancy rate (“DPR”) (percent), protein yield (“PROT”) (pounds), protein percentage (“PROTPCT”) (percent), and net merit (“NM”) (dollar). These traits are sex-limited, as no individual phenotypes can be measured on male animals. Instead, genetic merits of these traits defined as PTA (predicted transmitting ability) were estimated using phenotypes of all relatives. Most dairy bulls were progeny tested with a reasonably larger number of daughters (e.g., >50), and their PTA estimation is generally more or considerably more accurate than individual cow phenotype data. The genetic evaluation of traditional dairy traits of US Holstein population was performed quarterly by USDA. Detailed description of traits, genetic evaluation procedures, and genetic parameters used in the evaluation can be found at the USDA AIPL web site (http://aipl.arsusda.gov). It is meaningful to note that the dairy traits evaluated in this study are not independent: FAT and PROT are composite traits of MILK and FATPCT, and MILK and PROTPCT, respectively. NM is an index trait calculated based on protein yield, fat yield, production life, somatic cell score, daughter pregnancy, calving difficulty, and several type traits.

PTA data of all bulls with progeny testing data were downloaded from the USDA February 2007 genetic evaluation published at the AIPL site. The PTA data were analyzed using following two models:

yᵢⱼ=sᵢ+PTAdᵢⱼ [Equation 9]

yᵢ=β+β₁(SPTA)ᵢ+PTAdᵢ [Equation 10]

where yᵢ (yᵢⱼ) is the PTA of the i^{th} bull (PTA of the j^{th} son of the i^{th} sire); sᵢ is the effect of the i^{th} sire; (SPTA)ᵢ is the sire's PTA of the i^{th} bull of the whole sample; μ is the population mean; PTAdᵢ (PTAdᵢⱼ) is the residual bull PTA.

Equation 9 is referred to as the sire model, in which sires were fitted as fixed factors. Among all USA Holstein progeny tested bulls, a considerably large number of sires only have a very small number of progeny tested sons (e.g., some have one son), and it is clearly undesirable to fit sires as fixed factors in these cases. It is well known the USA Holstein herds have been making steady and rapid genetic progress in traditional dairy traits in the last several decades, implying that the sire's effect can be partially accounted for by fitting the birth year of a bull. For sires with <10 progeny tested sons, sires were replaced with son's birth year in Equation 9. Equation 10 is referred to as the SPTA model, in which sire's PTA are fitted as a covariate. Residual PTA (PTAdᵢ or PTAdᵢⱼ) were estimated using SAS PROC GLM procedure and used for further candidate gene analysis in this study.

Candidate gene interaction analysis. The association between SNP and residual PTA of each dairy trait was analyzed using the following linear models:

$${PTAd}_{i} = {\sum }_{j = 1}^{2} {\sum }_{k = 1}^{{n}_{gj}} {I}_{ijk} {β}_{jk} + {\sum }_{h = 1}^{{n}_{g 2}} {\sum }_{k = 1}^{{n}_{g 1}} {I}_{i 1 k} * {I}_{i 2 h} {δ}_{kh} + {e}_{i} \qquad [ Equation 11 ]$$

where PTAdᵢ is the preadjusted PTA of the i^{th} bull as defined in Equation 10 under the sire model and can be replaced with PTAdᵢ as defined in Equation 9 under the SPTA model; n_{gj} is the number of unordered genotypes at SNP j (j=1, 2); eᵢ is the residual effect; βₖ is the effect of genotype indicator Iᵢⱼₖ, and δₖₕ is the interaction effect between genotype indicator Iᵢ₁ₖ at the 1^{st} SNP and genotype indicator Iᵢ₂ₕ at the 2^{nd} SNP; and genotype indicator Iᵢⱼₖ is defined as

$${I}_{ijk} = \{ \begin{matrix}1 & if genotype being k at the jth SNP \\ 0 & otherwise\end{matrix} \qquad [ Equation 12 ]$$

Overall analyses consist of two steps. Original PTA data was first preadjusted using all bulls evaluated by USDA (Equation 9 and 10), and the preadjusted PTA was analyzed using Equation 11 for statistical associations between SNP and trait. The combination of Equations 9 and 11, and 10 and 11 was referred as to the sire model and the SPTA model, respectively.

Results of this analysis are shown in Table 1.

### Example 3

### Use of Single Nucleotide Polymorphisms (SNPs) to Improve Offspring Traits

To improve the average genetic merit of a population for a chosen trait, two or more of the markers with significant association to that trait can be used in selection of breeding animals. In the case of each discovered locus, use of animals possessing a marker allele (or a haplotype of multiple marker alleles) in population-wide LD with a favorable QTL allele will increase the breeding value of animals used in breeding, increase the frequency of that QTL allele in the population over time and thereby increase the average genetic merit of the population for that trait. This increased genetic merit can be disseminated to commercial populations for full realization of value.

For example, a progeny-testing scheme could greatly improve its rate of genetic progress or graduation success rate via the use of markers for screening juvenile bulls. Typically, a progeny testing program would use pedigree information and performance of relatives to select juvenile bulls as candidates for entry into the program with an accuracy of approx 0.5. However, by adding marker information, young bulls could be screened and selected with much higher accuracy. In this example, DNA samples from potential bull mothers and their male offspring could be screened with a genome-wide set of markers in linkage disequilibrium with QTL, and the bull-mother candidates with the best marker profile could be contracted for matings to specific bulls. If superovulation and embryo transfer (ET) is employed, a set of 5-10 offspring could be produced per bull mother per flush procedure. Then the marker set could again be used to select the best male offspring as a candidate for the progeny test program. If genome-wide markers are used, it was estimated that accuracies of marker selection could reach as high as 0.85 (Meuwissen et al., 2001). This additional accuracy could be used to greatly improve the genetic merit of candidates entering the progeny test program and thereby increasing the probability of successfully graduating a marketable progeny-tested bulls. This information could also be used to reduce program costs by decreasing the number of juvenile bull candidates tested while maintaining the same number of successful graduates. In the extreme, very accurate marker breeding values (MBV) could be used to directly market semen from juvenile sires without the need of progeny-testing at all. Due to the fact that juveniles could now be marketed starting at puberty instead of 4.5 to 5 years, generation interval could be reduced by more than half and rates of gain could increase as much as 68.3% (Schrooten et al., 2004). With the elimination of the need for progeny testing, the cost of genetic improvement for the artificial insemination industry would be vastly improved (Schaeffer, 2006).

In an alternate example, a centralized or dispersed genetic nucleus (GN) population of cattle could be maintained to produce juvenile bulls for use in progeny testing or direct sale on the basis of MBVs. A GN herd of 1000 cows could be expected to produce roughly 3000 offspring per year, assuming the top 10-15% of females were used as ET donors in a multiple-ovulation and embryo-transfer (MOET) scheme. However, markers could change the effectiveness MOET schemes and in vitro embryo production. Previously, MOET nucleus schemes have proven to be promising from the standpoint of extra genetic gain, but the costs of operating a nucleus herd together with the limited information on juvenile animals has limited widespread adoption. However, with marker information, juveniles can be selected much more accurately than before resulting in greatly reduced generation intervals and boosted rates of genetic response. This is especially true in MOET nucleus herd schemes because, previously, breeding values of full-sibs would be identical, but with marker information the best full-sib can be identified early in life. The marker information would also help limit inbreeding because less selection pressure would be placed on pedigree information and more on individual marker information. An early study (Meuwissen and van Arendonk, 1992) found advantages of up to 26% additional genetic gain when markers were employed in nucleus herd scenarios; whereas, the benefit in regular progeny testing was much less.

Together with MAS, female selection could also become an important source of genetic improvement particularly if markers explain substantial amounts of genetic variation. Further efficiencies could be gained by marker testing of embryos prior to implantation (Bredbacka, 2001). This would allow considerable selection to occur on embryos such that embryos with inferior marker profiles could be discarded prior to implantation and recipient costs. This would again increase the cost effectiveness of nucleus herds because embryo pre-selection would allow equal progress to be made with a smaller nucleus herd. Alternatively, this presents further opportunities for pre-selection prior to bulls entering progeny test and rates of genetic response predicted to be up to 31% faster than conventional progeny testing (Schrooten et al., 2004).

The first step in using a SNP for estimation of breeding value and selection in the GN is collection of DNA from all offspring that will be candidates for selection as breeders in the GN or as breeders in other commercial populations (in the present example, the 3,000 offspring produced in the GN each year). One method is to capture shortly after birth a small bit of ear tissue, hair sample, or blood from each calf into a labeled (bar-coded) tube. The DNA extracted from this tissue can be used to assay an essentially unlimited number of SNP markers and the results can be included in selection decisions before the animal reaches breeding age.

One method for incorporating into selection decisions the markers (or marker haplotypes) determined to be in population-wide LD with valuable QTL alleles (see Example 1) is based on classical quantitative genetics and selection index theory (Falconer and Mackay, 1996; Dekkers and Chakraborty, 2001). To estimate the effect of the marker in the population targeted for selection, a random sample of animals with phenotypic measurements for the trait of interest can be analyzed with a mixed animal model with the marker fitted as a fixed effect or as a covariate (regression of phenotype on number of allele copies). Results from either method of fitting marker effects can be used to derive the allele substitution effects, and in turn the breeding value of the marker:

α₁=q[a+d(q−p)] [Equation 13]

α₂=−p[a+d(q−p)] [Equation 14]

α=a+d(q−p) [Equation 15]

g_{A1A1}=2(α₁) [Equation 16]

g_{A1A2}=(α₁)+(α₂) [Equation 17]

g_{A2A2}=2(α₂) [Equation 18]

where α₁ and α₂ are the average effects of alleles 1 and 2, respectively; α is the average effect of allele substitution; p and q are the frequencies in the population of alleles 1 and 2, respectively; a and d are additive and dominance effects, respectively; g_{A1A1}, g_{A1A2} and g_{A2A2} are the (marker) breeding values for animals with marker genotypes A1A1, A1A2 and A2A2, respectively. The total trait breeding value for an animal is the sum of breeding values for each marker (or haplotype) considered and the residual polygenic breeding value:

EBVᵢⱼ=Σĝⱼ+Ûᵢ [Equation 19]

where EBVᵢⱼ is the Estimated Trait Breeding Value for the i^{th} animal, Σĝⱼ is the marker breeding value summed from j=1 to n where n is the total number of markers (haplotypes) under consideration, and Ûᵢ is the polygenic breeding value for the i^{th} animal after fitting the marker genotype(s).

These methods can readily be extended to estimate breeding values for selection candidates for multiple traits, the breeding value for each trait including information from multiple markers (haplotypes), all within the context of selection index theory and specific breeding objectives that set the relative importance of each trait. Other methods also exist for optimizing marker information in estimation of breeding values for multiple traits, including random models that account for recombination between markers and QTL (e.g., Fernando and Grossman, 1989), and the potential inclusion of all discovered marker information in whole-genome selection (Meuwissen et al., Genetics 2001). Through any of these methods, the markers reported herein that have been determined to be in population-wide LD with valuable QTL alleles may be used to provide greater accuracy of selection, greater rate of genetic improvement, and greater value accumulation in the dairy industry.

### Example 4

### Use of Multiple SNPs with Interaction Effects to Improve Offspring Traits

To illustrate the use of interaction effects in a breeding program, consider two causal mutations at two biallelic QTLs (denoted by A and B). Let A₁ and A₂, and B₁ and B₂ be the two alleles of QTL A and B, respectively. One way to model both interaction and main effects is to fit the effects of all genotype configurations:

yᵢ=Σβ(AₜAⱼ;BₛBₖ)I(AₜAⱼ;BₛBₖ)+aᵢ+εᵢ [Equation 20]

Where aᵢ denotes to a polygenic random effect; (AₜAⱼ; BₛBₖ) denotes to a genotype configuration consisting of genotypes at A and B; β(AₜAⱼ; BₛBₖ) is the regression coefficient for genotype configuration (AₜAⱼ; BₛBₖ); I(AₜAⱼ; BₛBₖ) is an index function defined as:

$$I ( {A}_{t} {A}_{j} ; {B}_{s} {B}_{k} ) = \{ \begin{matrix}1 & if genotype is ( {A}_{t} {A}_{j} ; {B}_{s} {B}_{k} ) \\ 0 & otherwise\end{matrix} \qquad [ Equation 21 ]$$

Equation [20] can be used for both detection and utilization of interaction effects. The effect of genotype configuration (AₜAⱼ; BₛBₖ) in Equation 20 can be fitted as fixed effects or a random effect.

The breeding value of a haplotype consisting of one allele from each QTL can be calculated using:

$$α ( {A}_{i} {B}_{j} ) = β ( {A}_{i} {A}_{1} ; {B}_{j} {B}_{1} ) f ( {A}_{1} {B}_{1} ) + β ( {A}_{i} {A}_{1} ; {B}_{j} {B}_{2} ) f ( {A}_{1} {B}_{2} ) + β ( {A}_{i} {A}_{2} ; {B}_{j} {B}_{1} ) f ( {A}_{2} {B}_{1} ) + β ( {A}_{i} {A}_{2} ; {B}_{j} {B}_{2} ) f ( {A}_{2} {B}_{2} ) \qquad [ Equation 22 ]$$

where f(AₖBₛ) (k, s=1, 2) represents the frequency of haplotype AₖBₛ. It should be noted that f(AₖBₛ) is not equal to the product of the corresponding allele frequency in presence of population-wise linkage disequilibrium.

The breeding value of an animal with genotype configuration (AᵢAⱼ; BₖBₛ) can be calculated as:

$$BV ( {A}_{i} {A}_{j} ; {B}_{k} {B}_{s} ) = 2 [ \begin{matrix}p ( {A}_{i} {B}_{k} ) α ( {A}_{i} {B}_{k} ) + \\ p ( {A}_{i} {B}_{s} ) α ( {A}_{i} {B}_{s} ) + \\ p ( {A}_{j} {B}_{k} ) α ( {A}_{j} {B}_{k} ) + \\ p ( {A}_{j} {B}_{s} ) α ( {A}_{j} {B}_{s} )\end{matrix} ] \qquad [ Equation 23 ]$$

where p(AᵢBⱼ) is the probability of a gamete produced by this animal having gamete haplotype AᵢBⱼ. It should be noted that the sum of probabilities of all possible haplotypes is equal to 1 and that the value of p(AᵢBⱼ) is a function of the recombination fraction between QTL A and B in case of a genotype configuration being heterozygous at both loci. To explain the linkage effect further, consider an animal with genotype A₁B₁/A₂B₂ (i.e. consisting of haplotypes A₁B₁ and A₂B₂). The probabilities of four different haplotypes for this animal can be calculated as

p(A₁B₁)=p(A₂B₂)=0.5(1−θ_{AB}) [Equation 24]

and

p(A₁B₂)=p(A₂B₁)=0.5θ_{AB} [Equation 25]

where θ_{AB} represents the recombination fraction between locus A and B.

The breeding value of a genotype configuration can be used for genetic improvement purpose in the same manner as the conventional polygenic breeding value.

It should be noted that the interaction effects can be estimated using various statistical models. It should also be noted that the above procedure can be easily extended for cases with multiple alleles and/or multiple loci (e.g., by including all possible genotype configurations in Equation 20).

### Example 5

### Identification of SNPs

A nucleic acid sequence contains a SNP of the present invention if it comprises at least 20 consecutive nucleotides that include and/or are adjacent to a polymorphism described in Tables 1 and 3 and the Sequence Listing. Alternatively, a SNP of the present invention may be identified by a shorter stretch of consecutive nucleotides which include or are adjacent to a polymorphism which is described in Tables 1 and 3 and the Sequence Listing in instances where the shorter sequence of consecutive nucleotides is unique in the bovine genome. A SNP site is usually characterized by the consensus sequence in which the polymorphic site is contained, the position of the polymorphic site, and the various alleles at the polymorphic site. “Consensus sequence” means DNA sequence constructed as the consensus at each nucleotide position of a cluster of aligned sequences. Such clusters are often used to identify SNP and Indel (insertion/deletion) polymorphisms in alleles at a locus. Consensus sequence can be based on either strand of DNA at the locus, and states the nucleotide base of either one of each SNP allele in the locus and the nucleotide bases of all Indels in the locus, or both SNP alleles using degenerate code (IUPAC code: M for A or C; R for A or G; W for A or T; S for C or G; Y for C or T; K for G or T; V for A or C or G; H for A or C or T; D for A or G or T; B for C or G or T; N for A or C or G or T; Additional code that we use include I for “-” or A; O for “-” or C; E for “-” or G; L for “-” or T; where “-” means a deletion). Thus, although a consensus sequence may not be a copy of an actual DNA sequence, a consensus sequence is useful for precisely designing primers and probes for actual polymorphisms in the locus.

Such SNP have a nucleic acid sequence having at least 90% sequence identity, more preferably at least 95% or even more preferably for some alleles at least 98% and in many cases at least 99% sequence identity, to the sequence of the same number of nucleotides in either strand of a segment of animal DNA which includes or is adjacent to the polymorphism. The nucleotide sequence of one strand of such a segment of animal DNA may be found in a sequence in the group consisting of SEQ ID NO:1 through SEQ ID NO:175. It is understood by the very nature of polymorphisms that for at least some alleles there will be no identity at the polymorphic site itself. Thus, sequence identity can be determined for sequence that is exclusive of the polymorphism sequence. The polymorphisms in each locus are described in Tables 1 and 3.

Shown below are examples of public bovine SNPs that match each other: SNP ss38333809 was determined to be the same as ss38333810 because 41 bases (with the polymorphic site at the middle) from each sequence match one another perfectly (match length=41, identity=100%).

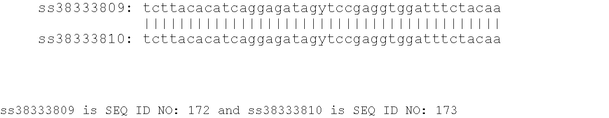

SNP ss38333809 was determined to be the same as ss38334335 because 41 bases (with the polymorphic site at the middle) from each sequence match one another at all bases except for one base (match length=41, identity=97%).

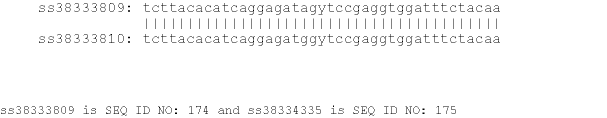

### Example 6

### Quantification of and Genetic Evaluation for Production Traits

Quantifying production traits can be accomplished by measuring milk of a cow and milk composition at each milking, or in certain time intervals only. In the USDA yield evaluation the milk production data are collected by Dairy Herd Improvement Associations (DHIA) using ICAR approved methods. Genetic evaluation includes all cows with the known sire and the first calving in 1960 and later and pedigree from birth year 1950 on. Lactations shorter than 305 days are extended to 305 days. All records are preadjusted for effects of age at calving, month of calving, times milked per day, previous days open, and heterogeneous variance. Genetic evaluation is conducted using the single-trait BLUP repeatability model. The model includes fixed effects of management group (herd×year×season plus register status), parity×age, and inbreeding, and random effects of permanent environment and herd by sire interaction. PTAs are estimated and published four times a year (February, May, August, and November). PTAs are calculated relative to a five year stepwise base i.e., as a difference from the average of all cows born in 2000. Bull PTAs are published estimating daughter performance for bulls having at least 10 daughters with valid lactation records.

### Example 7

### Quantification of Reproductive Traits in Daughters (Cows) and Sires' PTAs

Quantification of and genetic evaluation of the reproductive capability such as calving ease (CE), occurrence of stillbirths (SB) and daughter pregnancy rate (DPR). Calving ease measures the ability of a particular cow (daughter) to calve easily. CE is scored by the owner on a scale of 1 to 5, 1 meaning no problems encountered or unobserved birth and 5 meaning extreme difficulty. The CE PTAs for sires are expressed as percent difficult births in primiparous daughter heifers (% DBH), where difficult births are those scored as requiring considerable force or being extremely difficult (4 or 5 on a five point scale). SB is scored by the owner on a scale of 1 to 3, 1 meaning the calf was born alive and was alive 48 h postpartum, 2 meaning the calf was born dead, and 3 indicating the calf was born alive but died within 48 h postpartum. SB scores of 2 and 3 are combined into a single category for evaluation. The SB PTAs for sires are expressed as percent stillbirths in daughter heifers (% SBH), where stillborn calves are those scored as dead at birth or born alive but died within 48 h of birth (2 or 3 on a three point scale). Pregnancy rate is a function of the number of days open, which is the number of days between calving and a successful breeding. DPR is defined as the percentage of nonpregnant cows (daughters) that become pregnant during each 21-day period. A DPR PTA of “1” implies that daughters from this bull are 1% more likely to become pregnant during that estrus cycle than a bull with a DPR PTA of zero.

### Example 8

### Quantification of and Genetic Evaluation for Productive Life (PL)

Productive life (PL) is defined as the length of time a cow remains in a milking herd before removal by voluntary or involuntary culling (due to health or fertility problems), or death. PL is usually measured as the number of days, months, or days in milk (DIM) from the first calving to the day the cow exits the herd (due to death, culling, or selling to non-dairy purposes). Because some cows are still alive at the time of data collection, their records are projected (VanRaden, P. M. and E. J. H. Klaaskate. 1993) or treated as censored (Ducrocq, 1987). The USDA genetic evaluation for PL includes all cows with first calving in 1960 and later (born in 1950 and later for the pedigree). Cows born at least 3 years prior to evaluation, with a valid sire ID and first lactation records are considered. PL is considered to be completed at 7 years of age. Records are extended for cows that have not had the opportunity to reach 7 years of age because they are still alive, were sold for dairy purposes, or the herd discontinued testing. Cows sold for dairy purposes or in herds that discontinued testing receive extended records if they had opportunity to reach 3 years of age; otherwise their records are discarded. The method of genetic evaluation is a single trait BLUP animal model. The statistical model includes effects of management group (based on herd of first lactation and birth date) and sire by herd interaction. Sires' PTAs for PL are calculated relative to a five year stepwise base i.e., as a difference from the average PL of all cows born in 2000.

### Example 9

### Quantification of Somatic Cell Score in Daughters (Cows) and Sires' PTAs

Quantifying somatic cell score (SCS) is accomplished by calculating log₂ (SCC/100,000)+3, where SCC is number of somatic cells per milliliter of milk from a cow (daughter). The SCS PTAs for sires are expressed as a deviation from a SCS PTA of zero.

### Example 10

### Discovery of Novel Associations of SNPs within Candidate Genes

Animal sample and genotyping. A total of 3145 Holstein bulls with a NAAB code were downloaded from USDA AIPL web site (http://aipl.arsusda.gov) to form a resource population for this study. A total of 22 SNPs (single nucleotide polymorphisms) from 10 candidate genes (leptin, pou1F1, kappa casein, osteopontin, beta2-adrenergic receptor, growth hormone receptor, proteinase inhibitor, breast cancer resistance protein, diacylglycerol acyltransferase) were genotyped internally using the ABI Taqman platform or externally (Genaissance Pharmaceuticals, Inc., New Haven, Conn.) using various chemistries.

All SNPs used in this study have two alleles, resulting in a total of three unordered genotypes for each SNP (two homozygotes and one heterozygote). If <300 bulls are homozygous for the minor allele, the minor allele homozygote class can be merged with the heterozygote to form a composite genotype (genotype iiij is denoted to both genotype ii and ij) or excluded from analyses. Consequently, analyses can be performed using original genotypes, composite genotypes, and data that excludes the least frequent genotype when the number of bulls with least frequent genotype is smaller than 300.

Trait phenotype preadjustment. Analyzed traits include milk yield (“MILK”) (pounds), fat yield (“FAT”) (pounds), fat percentage (“FATPCT”) (percent), productive life (“PL”) (months), somatic cell score (“SCS”) (Log), daughter pregnancy rate (“DPR”) (percent), protein yield (“PROT”) (pounds), protein percentage (“PROTPCT”) (percent), and net merit (“NM”) (dollar). These traits are sex-limited, so genetic merits of these traits are defined as PTA (predicted transmitting ability) and were estimated using phenotypes of all relatives. Detailed description of traits, genetic evaluation procedures, and genetic parameters used in the evaluation can be found at the USDA AIPL web site (http://aipl.arsusda.gov). It is meaningful to note that the dairy traits evaluated in this study are not independent.

PTA data of all bulls with progeny testing data were downloaded from the USDA evaluation published at the AIPL site in November, 2005. The PTA data were analyzed using the following two models:

yᵢⱼ=sᵢ+PTAdᵢⱼ [Equation 26]

yᵢ=μ+β₁(SPTA)ᵢ+PTAdᵢ [Equation 27]

where yᵢ (yᵢⱼ) is the PTA of the i^{th} bull (PTA of the j^{th} son of the i^{th} sire); sᵢ is the effect of the i^{th} sire; (SPTA)ᵢ is the sire's PTA of the i^{th} bull of the whole sample; μ is the population mean; PTAdᵢ (PTAdᵢⱼ) is the residual bull PTA.

Equation 26 is referred to as the sire model, in which sires were fitted as fixed factors. For sires with <10 progeny tested sons, sires were replaced with son's birth year in Equation 26. Equation 27 is referred to as the SPTA model, in which sire's PTA are fitted as a covariate. Residual PTA (PTAdᵢ or PTAdᵢⱼ) were estimated using SAS PROC GLM procedure and used for further candidate gene analysis in this study.

Candidate gene analysis. The association between SNP and residual PTA of each dairy trait was analyzed using the following linear models:

$$\begin{matrix}{PTAd}_{i} = μ + {β}_{1} {x}_{i} + {e}_{i} & [ Equation 28 ] \\ {PTAd}_{i} = {\sum }_{k = 1}^{{n}_{g }} {I}_{ik} {β}_{k} + {e}_{i} & [ Equation 29 ]\end{matrix}$$

where PTAdᵢ is the preadjusted PTA of the i^{th} bull as defined in Equation 26 under the sire model and can be replaced with PTAdᵢ as defined in Equation 27 under the SPTA model; xᵢ is the number of copies of a specific SNP allele that the i^{th} bull has, and β₂ is the regression coefficient for xᵢ; n_{g} is the number of unordered genotypes; eᵢ is the residual effect; and βₖ is the effect of genotype indicator Iᵢₖ that is defined as

$${I}_{ik} = \{ \begin{matrix}1 & if genotype being k \\ 0 & otherwise\end{matrix} \qquad [ Equation 30 ]$$

Overall analyses consist of two steps. Original PTA data was first preadjusted using all bulls evaluated by USDA (Equations 26 and 27), and the preadjusted PTA was analyzed using Equations 28 and 29 for statistical associations between SNP and trait. The combination of Equations 26 and 28, 26 and 29, 27 and 28, and 27 and 29 was referred as to the sire_allele, sire_genotype, SPTA_allele, SPTA_genotype model, respectively.

The effect of a SNP on a trait was described by additive (=(Gᵢᵢ−Gⱼⱼ)/2), dominance (=Gᵢⱼ−(Gᵢᵢ−Gⱼⱼ)/2), or difference between two genotype (Gᵢⱼ−Gⱼⱼ), where i, and j denote the two alleles of the SNP, and Gᵢⱼ represents the mean of genotype ij.

Results of this analysis are shown in Table 1 and the Sequence Listing. Abbreviations for traits include the following: Fitness traits including pregnancy rate (PR), daughter pregnancy rate (DPR), productive life (PL), somatic cell count (SCC) and somatic cell score (SCS); and productivity traits including total milk yield (MY), milk fat percentage (FP), milk fat yield (FY), milk protein percentage (PP), milk protein yield (PY), total lifetime production (PL); and Net Merit (NM).

TABLE 1
The following table describes genes, markers, trait associations, and interactions effects
resulting from the experiments described herein.

| | | SEQ_ID | | | SEQ_ID | |
|---|---|---|---|---|---|---|
| | | For Marker | | | For | ASSOCIATED |
| GENE_1 | Marker 1 | 1* | GENE_2 | Marker 2 | Marker 2* | TRAITS |
| ADRB2 | NBQA_00015 | 15 | SPP1 | NBGA_00003 | 2 | SCS |
| ADRB2 | NBQA_00015 | 15 | LEP | NBQA_00011 | 13 | FY, NM, PY |
| ADRB2 | NBQA_00015 | 15 | GHR | NBQA_00006 | 9 | DPR, PL |
| ADRB2 | NBQA_00015 | 15 | DGAT1 | NBGA_00001 | 1 | NM, PY |
| ADRB2 | NBQA_00016 | 16 | LEP | NBQA_00017 | 17 | PL |
| ADRB2 | NBQA_00016 | 16 | LEP | NBQA_00009 | 11 | PL |
| ADRB2 | NBQA_00016 | 16 | LEP | NBQA_00001 | 5 | PL |
| ADRB2 | NBQA_00016 | 16 | DGAT1 | NBGA_00001 | 1 | DPR, FP, PL, |
| | | | | | | PY |
| CATSPER | bCATSPER_A250G | 20 | n/a | n/a | n/a | DPR |
| CATSPER | bCATSPER_C562A | 23 | n/a | n/a | n/a | DPR |
| CD14 | bCD14_C-5T | 31 | n/a | n/a | n/a | DPR, FY, PL |
| CD14 | bCD14_A523G | 29 | n/a | n/a | n/a | PY |
| CSN3 | NBQA_00012 | 14 | n/a | n/a | n/a | PL |
| CSN3 | NBQA_00012 | 14 | SPP1 | NBGA_00003 | 2 | FP, MY, PP |
| CSN3 | NBQA_00012 | 14 | GHR | NBQA_00005 | 8 | FP, MY, PP |
| CSN3 | NBQA_00012 | 14 | PI | NBQA_00004 | 7 | NM, PY |
| CSN3 | NBQA_00012 | 14 | POU1F1 | NBQA_00003 | 6 | DPR, FP, PP |
| CSN3 | NBQA_00012 | 14 | DGAT1 | NBGA_00001 | 1 | FY |
| DGAT1 | NBGA_00001 | 1 | n/a | n/a | n/a | DPR, PL |
| DGAT1 | NBGA_00001 | 1 | SPP1 | NBGA_00003 | 2 | NM, PL |
| DGAT1 | NBGA_00001 | 1 | GHR | NBQA_00006 | 9 | PP |
| DGAT1 | NBGA_00001 | 1 | POU1F1 | NBQA_00003 | 6 | FY, MY, NM, |
| | | | | | | PY |
| GHR | NBQA_00005 | 8 | n/a | n/a | n/a | SCS |
| GHR | NBQA_00005 | 8 | LEP | NBQA_00017 | 17 | PL |
| GHR | NBQA_00005 | 8 | PI | NBGA_00005 | 4 | MY, PY |
| GHR | NBQA_00005 | 8 | LEP | NBQA_00009 | 11 | PL |
| GHR | NBQA_00005 | 8 | LEP | NBQA_00001 | 5 | PL |
| GHR | NBQA_00006 | 9 | n/a | n/a | n/a | DPR |
| GHR | NBQA_00006 | 9 | LEP | NBQA_00011 | 13 | PP, PL |
| GHR | NBQA_00018 | 18 | n/a | n/a | n/a | DPR |
| GHR | NBQA_00018 | 18 | SPP1 | NBGA_00003 | 2 | PL, PP |
| GHR | NBQA_00018 | 18 | LEP | NBQA_00011 | 13 | NM, PL |
| IGF2R | bIGF2R_T6569C | 71 | n/a | n/a | n/a | DPR, FY |
| LEP | NBQA_00001 | 5 | n/a | n/a | n/a | PL |
| LEP | NBQA_00009 | 11 | n/a | n/a | n/a | PL |
| LEP | NBQA_00011 | 13 | SPP1 | NBGA_00003 | 2 | SCS |
| LEP | NBQA_00011 | 13 | PI | NBQA_00010 | 12 | MY, NM, PL, |
| | | | | | | PY |
| LEP | NBQA_00017 | 17 | n/a | n/a | n/a | PL |
| LIF | bLIF_G884A | 82 | n/a | n/a | n/a | FP, PL |
| LIF | bLIF_G972T | 83 | n/a | n/a | n/a | DPR, PL |
| LIF | bLIF_A1093G | 79 | n/a | n/a | n/a | FY |
| OSM | bOSM_A290G | 84 | n/a | n/a | n/a | FY |
| PI | NBQA_00010 | 12 | n/a | n/a | n/a | DPR |
| PI | NBQA_00010 | 12 | SPP1 | NBGA_00003 | 2 | FP, MY, PP |
| PI | NBGA_00004 | 3 | n/a | n/a | n/a | DPR |
| PI | NBGA_00004 | 3 | SPP1 | NBGA_00003 | 2 | FP, MY, PP |
| PI | NBQA_00004 | 7 | SPP1 | NBGA_00003 | 2 | FP, PP |
| PI | NBQA_00007 | 10 | SPP1 | NBGA_00003 | 2 | FP, PP |
| PI | NBGA_00005 | 4 | SPP1 | NBGA_00003 | 2 | FP, MY, PY |
| POU1F1 | NBQA_00003 | 6 | n/a | n/a | n/a | PL, SCS |
| POU1F1 | NBQA_00003 | 6 | SPP1 | NBGA_00003 | 2 | FY, SCS |
| RCN3 | bRCN3_CG_143 | 87 | n/a | n/a | n/a | DPR, PY |
| RIM2 | bRIM2_G5152A | 103 | n/a | n/a | n/a | DPR, SCS |
| SPP1 | NBGA_00003 | 2 | n/a | n/a | n/a | DPR, PL, SCS |
| TLE4 | bTLE4_G611A | 139 | n/a | n/a | n/a | MY, NM, PL, |
| | | | | | | PY |

*Details for each polymorphism including location, length, SEQ ID number, and alleles, are located in Table 4 and the sequence listing.

### Example 11

### Discovery of New Markers in the CATSPER, CD14, IGF2R, LIF, OSM, RCN3, RIM2, and TLE4 Genes and Association with Dairy Productivity Traits

A whole-genome scan was conducted using 3000 Holstein bulls to identify quantitative trait loci (QTL) for dairy productivity traits on all bovine chromosomes. This invention concerns QTL (and selected candidate genes) on chromosomes BTA07 (CD14), BTA08 (TLE4) BTA09 (IGF2R), BTA14 (RIM2), BTA17 (LIF, OSM), BTA18 (RCN3), and BTA29 (CATSPER). Flanking sequences of the SNPs used in the whole-genome scan that were found to be associated with dairy productivity traits were used to BLAST against the public bovine genome sequence assembly. Genes were identified proximal and distal (within ˜5 cM) to the QTL SNP location and researched to determine putative function. For selected QTL on chromosomes BTA07, BTA08, BTA09, BTA14, BTA17, BTA18, and BTA29 candidate genes, CD14, TLE4, IGF2R, RIM2, LIF and OSM, RCN3, and CATSPER, respectively, were chosen for novel marker discovery. Gene and NCBI GeneID numbers can are shown in Table 2 below (www.ncbi.nlm.nih.gov/sites/entrez?db=Gene).

TABLE 2
The following table describes genes
correlated NCBI GeneID numbers.

| Gene | NCBI GeneID |
|---|---|
| CATSPER | 523556 |
| CD14 | 281048 |
| IGF2R | 281849 |
| LIF | 280840 |
| OSM | 319086 |
| RCN3 | 522073 |
| RIM2 | 535674 |
| TLE4 | 508893 |
| ABCG2 | 536203 |
| ADRB2 | 281605 |
| CSN3 | 281728 |
| DGAT1 | 282609 |
| GHR | 280805 |
| LEP | 280836 |
| PI | 280699 |
| POU1F1 | 282315 |
| SPP1 | 281499 |

A total of 23 Holstein bulls, selected from the 3000 used for the whole-genome scan, were used as a discovery panel to identify novel genetic markers (SNPs and insertion-deletions, or INDELs) by sequencing the candidate genes and comparing forward and reverse strand sequences between all 23 samples. All Holstein DNA was extracted from semen using standard protocols. Standard laboratory PCR was used to amplify DNA fragments containing the coding region and regulatory regions of the genes for sequencing. Standard direct PCR product sequencing was conducted and resolved on an ABI 3730×1 Automated Sequencer (Applied Biosystems, Foster City, Calif.).

To perform association analysis, genetic markers discovered in candidate genes using the panel of 23 Holsteins were genotyped by sequencing on a panel of 108 additional Holsteins (with 88 selected from the 3000 used in the whole-genome scan and 20 unique to the 108 animal panel). Genotypes of the 108 animal panel were combined with the genotypes from the 23 animal discovery panel for a total of 131 genotypes per genetic marker. Association analysis was carried as described above.

This experiment resulted in number confirmed associations in and around CD14, TLE4, IGF2R, RIM2, LIF and OSM, RCN3, and CATSPER as well as the identification of a large number of SNPs. Results of the association study are further described in Tables 1 and the Sequence Listing, and novel polymorphisms are identified in Tables 3 and the Sequence listing. In each case, details regarding the location, length, and alleles for each polymorphism are described in Table 4.

TABLE 3
The following table includes a list of novel markers, gene names,
and SEQ ID numbers resulting from the experiment described above.

| GENE | Marker Name | SEQ_ID* |
|---|---|---|
| CATSPER | bCATSPER_CT_238 | 24 |
| CATSPER | bCATSPER_TC_275 | 19 |
| CATSPER | bCATSPER_A250G | 20 |
| CATSPER | bCATSPER_A514T | 21 |
| CATSPER | bCATSPER_C562A | 23 |
| CATSPER | bCATSPER_CT_376 | 25 |
| CATSPER | bCATSPER_GA_38 | 26 |
| CATSPER | bCATSPER_AG_176 | 22 |
| CD14 | bCD14_C-5T | 31 |
| CD14 | bCD14_A439C | 28 |
| CD14 | bCD14_A523G | 29 |
| CD14 | bCD14_A933G | 30 |
| CD14 | bCD14_A1216G | 27 |
| CD14 | bCD14_T1236G | 32 |
| IGF2R | bIGF2R_GA_444 | 60 |
| IGF2R | bIGF2R_GA_167 | 50 |
| IGF2R | bIGF2R_AG_448 | 37 |
| IGF2R | bIGF2R_T2898C | 67 |
| IGF2R | bIGF2R_T5091C | 70 |
| IGF2R | bIGF2R_CT_365 | 44 |
| IGF2R | bIGF2R_I1_77 | 65 |
| IGF2R | bIGF2R_GC_54 | 62 |
| IGF2R | bIGF2R_TG_151 | 77 |
| IGF2R | bIGF2R_TC_107 | 72 |
| IGF2R | bIGF2R_CA_173 | 39 |
| IGF2R | bIGF2R_CT_541 | 47 |
| IGF2R | bIGF2R_GT_125 | 63 |
| IGF2R | bIGF2R_GA_115 | 49 |
| IGF2R | bIGF2R_GA_92 | 61 |
| IGF2R | bIGF2R_AG_228 | 35 |
| IGF2R | bIGF2R_GA_199 | 51 |
| IGF2R | bIGF2R_GA_363 | 56 |
| IGF2R | bIGF2R_T3526C | 68 |
| IGF2R | bIGF2R_AG_103 | 33 |
| IGF2R | bIGF2R_T3975C | 69 |
| IGF2R | bIGF2R_CT_338 | 42 |
| IGF2R | bIGF2R_TC_348 | 75 |
| IGF2R | bIGF2R_AG_280 | 36 |
| IGF2R | bIGF2R_CT_489 | 46 |
| IGF2R | bIGF2R_CG_42 | 40 |
| IGF2R | bIGF2R_GA_364 | 57 |
| IGF2R | bIGF2R_CT_387 | 45 |
| IGF2R | bIGF2R_TC_287 | 74 |
| IGF2R | bIGF2R_TC_358 | 76 |
| IGF2R | bIGF2R_CT_349 | 43 |
| IGF2R | bIGF2R_GA_201 | 52 |
| IGF2R | bIGF2R_CT_239 | 41 |
| IGF2R | bIGF2R_C5748T | 38 |
| IGF2R | bIGF2R_GA_310 | 54 |
| IGF2R | bIGF2R_GA_408 | 58 |
| IGF2R | bIGF2R_GA_433 | 59 |
| IGF2R | bIGF2R_AG_104 | 34 |
| IGF2R | bIGF2R_GA_114 | 48 |
| IGF2R | bIGF2R_GA_332 | 55 |
| IGF2R | bIGF2R_T6569C | 71 |
| IGF2R | bIGF2R_GA_218 | 53 |
| IGF2R | bIGF2R_TC_221 | 73 |
| IGF2R | bIGF2R_I1_407 | 64 |
| IGF2R | bIGF2R_I2_263 | 66 |
| IGF2R | bIGF2R_TG_460 | 78 |
| LIF | bLIF_C393T | 81 |
| LIF | bLIF_G884A | 82 |
| LIF | bLIF_G972T | 83 |
| LIF | bLIF_A1093G | 79 |
| LIF | bLIF_C1613T | 80 |
| OSM | bOSM_A290G | 84 |
| OSM | bOSM_G662A | 85 |
| RCN3 | bRCN3_CT_347 | 90 |
| RCN3 | bRCN3_CT_248 | 88 |
| RCN3 | bRCN3_TC_173 | 91 |
| RCN3 | bRCN3_A574G | 86 |
| RCN3 | bRCN3_CT_287 | 89 |
| RCN3 | bRCN3_CG_143 | 87 |
| RIM2 | bRIM2_AG_124 | 92 |
| RIM2 | bRIM2_CT_531 | 99 |
| RIM2 | bRIM2_CT_699 | 100 |
| RIM2 | bRIM2_CT_376 | 97 |
| RIM2 | bRIM2_AG_347 | 94 |
| RIM2 | bRIM2_GA_140 | 105 |
| RIM2 | bRIM2_AG_153 | 93 |
| RIM2 | bRIM2_GT_149 | 107 |
| RIM2 | bRIM2_TC_230 | 109 |
| RIM2 | bRIM2_TG_667 | 112 |
| RIM2 | bRIM2_GT_99 | 108 |
| RIM2 | bRIM2_GA_125 | 104 |
| RIM2 | bRIM2_C2963G | 95 |
| RIM2 | bRIM2_TC_360 | 110 |
| RIM2 | bRIM2_CT_121 | 96 |
| RIM2 | bRIM2_CT_442 | 98 |
| RIM2 | bRIM2_TG_472 | 111 |
| RIM2 | bRIM2_GA_494 | 106 |
| RIM2 | bRIM2_G5152A | 103 |
| RIM2 | bRIM2_D1_421 | 101 |
| RIM2 | bRIM2_D2_85 | 102 |
| TLE4 | bTLE4_TG_251 | 170 |
| TLE4 | bTLE4_TC_200 | 162 |
| TLE4 | bTLE4_AC_114 | 115 |
| TLE4 | bTLE4_TC_149 | 160 |
| TLE4 | bTLE4_TC_79 | 168 |
| TLE4 | bTLE4_AG_212 | 118 |
| TLE4 | bTLE4_AG_458 | 121 |
| TLE4 | bTLE4_AT_152 | 123 |
| TLE4 | bTLE4_C453T | 126 |
| TLE4 | bTLE4_G358T | 137 |
| TLE4 | bTLE4_T475C | 155 |
| TLE4 | bTLE4_GA_102 | 143 |
| TLE4 | bTLE4_TC_319 | 165 |
| TLE4 | bTLE4_AC_108 | 114 |
| TLE4 | bTLE4_CG_116 | 128 |
| TLE4 | bTLE4_GA_205 | 145 |
| TLE4 | bTLE4_GC_374 | 149 |
| TLE4 | bTLE4_GT_382 | 152 |
| TLE4 | bTLE4_TA_247 | 157 |
| TLE4 | bTLE4_GT_248 | 150 |
| TLE4 | bTLE4_TC_276 | 163 |
| TLE4 | bTLE4_TC_353 | 166 |
| TLE4 | bTLE4_AG_89 | 122 |
| TLE4 | bTLE4_TG_132 | 169 |
| TLE4 | bTLE4_C563A | 127 |
| TLE4 | bTLE4_G611A | 139 |
| TLE4 | bTLE4_TC_198 | 161 |
| TLE4 | bTLE4_G848A | 141 |
| TLE4 | bTLE4_G913C | 142 |
| TLE4 | bTLE4_A988G | 113 |
| TLE4 | bTLE4_C1072T | 125 |
| TLE4 | bTLE4_T1215C | 154 |
| TLE4 | bTLE4_TC_315 | 164 |
| TLE4 | bTLE4_TA_328 | 159 |
| TLE4 | bTLE4_CT_96 | 133 |
| TLE4 | bTLE4_GA_107 | 144 |
| TLE4 | bTLE4_GT_365 | 151 |
| TLE4 | bTLE4_CT_167 | 130 |
| TLE4 | bTLE4_TC_423 | 167 |
| TLE4 | bTLE4_AG_161 | 117 |
| TLE4 | bTLE4_AG_307 | 120 |
| TLE4 | bTLE4_CT_480 | 131 |
| TLE4 | bTLE4_AG_260 | 119 |
| TLE4 | bTLE4_TA_291 | 158 |
| TLE4 | bTLE4_CG_414 | 129 |
| TLE4 | bTLE4_AG_134 | 116 |
| TLE4 | bTLE4_G750A | 140 |
| TLE4 | bTLE4_GC_199 | 148 |
| TLE4 | bTLE4_AT_262 | 124 |
| TLE4 | bTLE4_GA_568 | 146 |
| TLE4 | bTLE4_TA_141 | 156 |
| TLE4 | bTLE4_TG_571 | 171 |
| TLE4 | bTLE4_CT_627 | 132 |
| TLE4 | bTLE4_GA_66 | 147 |
| TLE4 | bTLE4_G560A | 138 |
| TLE4 | bTLE4_I51_7 | 153 |
| TLE4 | bTLE4_D615_2 | 136 |
| TLE4 | bTLE4_D296_2 | 134 |
| TLE4 | bTLE4_D393_1 | 135 |

*Details for each polymorphism including location, SEQ ID number, and alleles, are located in Table 4 and the sequence listing.

TABLE 4
The following table describes the polymorphisms listed in Tables 1 and 3 in more detail,
including the SEQ ID number, polymorphism Position, and Alleles.

| | | | Polymorphism | Polymorphism | | |
|---|---|---|---|---|---|---|
| SEQ_ID | GENE | Marker Name | Start | End | ALLELE1 | ALLELE2 |
| 1 | DGAT1 | NBGA_00001 | 308 | 309 | AA | GC |
| 2 | SPP1 | NBGA_00003 | 307 | 307 | T | — |
| 3 | PI | NBGA_00004 | 63 | 63 | C | T |
| 4 | PI | NBGA_00005 | 232 | 232 | C | T |
| 5 | LEP | NBQA_00001 | 306 | 306 | C | G |
| 6 | POU1F1 | NBQA_00003 | 240 | 240 | A | G |
| 7 | PI | NBQA_00004 | 198 | 198 | A | G |
| 8 | GHR | NBQA_00005 | 244 | 244 | A | T |
| 9 | GHR | NBQA_00006 | 365 | 365 | G | T |
| 10 | PI | NBQA_00007 | 81 | 81 | C | G |
| 11 | LEP | NBQA_00009 | 247 | 247 | A | G |
| 12 | PI | NBQA_00010 | 78 | 78 | G | T |
| 13 | LEP | NBQA_00011 | 214 | 214 | A | G |
| 14 | CSN3 | NBQA_00012 | 37 | 37 | A | C |
| 15 | ADRB2 | NBQA_00015 | 1247 | 1247 | G | T |
| 16 | ADRB2 | NBQA_00016 | 692 | 692 | A | C |
| 17 | LEP | NBQA_00017 | 176 | 176 | A | G |
| 18 | GHR | NBQA_00018 | 276 | 276 | A | G |
| 19 | CATSPER | bCATPSER_TC_275 | 72 | 72 | T | C |
| 20 | CATSPER | bCATSPER_A250G | 72 | 72 | A | G |
| 21 | CATSPER | bCATSPER_A514T | 72 | 72 | A | T |
| 22 | CATSPER | bCATSPER_AG_176 | 72 | 72 | A | G |
| 23 | CATSPER | bCATSPER_C562A | 72 | 72 | C | A |
| 24 | CATSPER | bCATSPER_CT_238 | 72 | 72 | C | T |
| 25 | CATSPER | bCATSPER_CT_376 | 72 | 72 | C | T |
| 26 | CATSPER | bCATSPER_GA_38 | 72 | 72 | G | A |
| 27 | CD14 | bCD14_A1216G | 72 | 72 | A | G |
| 28 | CD14 | bCD14_A439C | 72 | 72 | A | C |
| 29 | CD14 | bCD14_A523G | 72 | 72 | A | G |
| 30 | CD14 | bCD14_A933G | 72 | 72 | A | G |
| 31 | CD14 | bCD14_C-5T | 72 | 72 | C | T |
| 32 | CD14 | bCD14_T1236G | 72 | 72 | T | G |
| 33 | IGF2R | bIGF2R_AG_103 | 72 | 72 | A | G |
| 34 | IGF2R | bIGF2R_AG_104 | 72 | 72 | A | G |
| 35 | IGF2R | bIGF2R_AG_228 | 72 | 72 | A | G |
| 36 | IGF2R | bIGF2R_AG_280 | 72 | 72 | A | G |
| 37 | IGF2R | bIGF2R_AG_448 | 72 | 72 | A | G |
| 38 | IGF2R | bIGF2R_C5748T | 72 | 72 | C | T |
| 39 | IGF2R | bIGF2R_CA_173 | 72 | 72 | C | A |
| 40 | IGF2R | bIGF2R_CG_42 | 72 | 72 | C | G |
| 41 | IGF2R | bIGF2R_CT_239 | 72 | 72 | C | T |
| 42 | IGF2R | bIGF2R_CT_338 | 72 | 72 | C | T |
| 43 | IGF2R | bIGF2R_CT_349 | 72 | 72 | C | T |
| 44 | IGF2R | bIGF2R_CT_365 | 72 | 72 | C | T |
| 45 | IGF2R | bIGF2R_CT_387 | 72 | 72 | C | T |
| 46 | IGF2R | bIGF2R_CT_489 | 72 | 72 | C | T |
| 47 | IGF2R | bIGF2R_CT_541 | 72 | 72 | C | T |
| 48 | IGF2R | bIGF2R_GA_114 | 72 | 72 | G | A |
| 49 | IGF2R | bIGF2R_GA_115 | 72 | 72 | G | A |
| 50 | IGF2R | bIGF2R_GA_167 | 72 | 72 | G | A |
| 51 | IGF2R | bIGF2R_GA_199 | 72 | 72 | G | A |
| 52 | IGF2R | bIGF2R_GA_201 | 72 | 72 | G | A |
| 53 | IGF2R | bIGF2R_GA_218 | 72 | 72 | G | A |
| 54 | IGF2R | bIGF2R_GA_310 | 72 | 72 | G | A |
| 55 | IGF2R | bIGF2R_GA_332 | 72 | 72 | G | A |
| 56 | IGF2R | bIGF2R_GA_363 | 72 | 72 | G | A |
| 57 | IGF2R | bIGF2R_GA_364 | 72 | 72 | G | A |
| 58 | IGF2R | bIGF2R_GA_408 | 72 | 72 | G | A |
| 59 | IGF2R | bIGF2R_GA_433 | 72 | 72 | G | A |
| 60 | IGF2R | bIGF2R_GA_444 | 72 | 72 | G | A |
| 61 | IGF2R | bIGF2R_GA_92 | 72 | 72 | G | A |
| 62 | IGF2R | bIGF2R_GC_54 | 72 | 72 | G | C |
| 63 | IGF2R | bIGF2R_GT_125 | 72 | 72 | G | T |
| 64 | IGF2R | bIGF2R_I1_407 | 72 | 72 | A | — |
| 65 | IGF2R | bIGF2R_I1_77 | 72 | 72 | T | — |
| 66 | IGF2R | bIGF2R_I2_263 | 72 | 73 | CC | — |
| 67 | IGF2R | bIGF2R_T2898C | 72 | 72 | T | C |
| 68 | IGF2R | bIGF2R_T3526C | 72 | 72 | T | C |
| 69 | IGF2R | bIGF2R_T3975C | 72 | 72 | T | C |
| 70 | IGF2R | bIGF2R_T5091C | 72 | 72 | T | C |
| 71 | IGF2R | bIGF2R_T6569C | 72 | 72 | T | C |
| 72 | IGF2R | bIGF2R_TC_107 | 72 | 72 | T | C |
| 73 | IGF2R | bIGF2R_TC_221 | 72 | 72 | T | C |
| 74 | IGF2R | bIGF2R_TC_287 | 72 | 72 | T | C |
| 75 | IGF2R | bIGF2R_TC_348 | 72 | 72 | T | C |
| 76 | IGF2R | bIGF2R_TC_358 | 72 | 72 | T | C |
| 77 | IGF2R | bIGF2R_TG_151 | 72 | 72 | T | G |
| 78 | IGF2R | bIGF2R_TG_460 | 72 | 72 | T | G |
| 79 | LIF | bLIF_A1093G | 72 | 72 | A | G |
| 80 | LIF | bLIF_C1613T | 72 | 72 | C | T |
| 81 | LIF | bLIF_C393T | 72 | 72 | C | T |
| 82 | LIF | bLIF_G884A | 72 | 72 | G | A |
| 83 | LIF | bLIF_G972T | 72 | 72 | G | T |
| 84 | OSM | bOSM_A290G | 72 | 72 | A | G |
| 85 | OSM | bOSM_G662A | 72 | 72 | G | A |
| 86 | RCN3 | bRCN3_A574G | 72 | 72 | A | G |
| 87 | RCN3 | bRCN3_CG_143 | 72 | 72 | C | G |
| 88 | RCN3 | bRCN3_CT_248 | 72 | 72 | C | T |
| 89 | RCN3 | bRCN3_CT_287 | 72 | 72 | C | T |
| 90 | RCN3 | bRCN3_CT_347 | 72 | 72 | C | T |
| 91 | RCN3 | bRCN3_TC_173 | 72 | 72 | T | C |
| 92 | RIM2 | bRIM2_AG_124 | 72 | 72 | A | G |
| 93 | RIM2 | bRIM2_AG_153 | 72 | 72 | A | G |
| 94 | RIM2 | bRIM2_AG_347 | 72 | 72 | A | G |
| 95 | RIM2 | bRIM2_C2963G | 72 | 72 | C | G |
| 96 | RIM2 | bRIM2_CT_121 | 72 | 72 | C | T |
| 97 | RIM2 | bRIM2_CT_376 | 72 | 72 | C | T |
| 98 | RIM2 | bRIM2_CT_442 | 72 | 72 | C | T |
| 99 | RIM2 | bRIM2_CT_531 | 72 | 72 | C | T |
| 100 | RIM2 | bRIM2_CT_699 | 72 | 72 | C | T |
| 101 | RIM2 | bRIM2_D1_421 | 72 | 72 | G | — |
| 102 | RIM2 | bRIM2_D2_85 | 72 | 73 | TC | — |
| 103 | RIM2 | bRIM2_G5152A | 72 | 72 | G | A |
| 104 | RIM2 | bRIM2_GA_125 | 72 | 72 | G | A |
| 105 | RIM2 | bRIM2_GA_140 | 72 | 72 | G | A |
| 106 | RIM2 | bRIM2_GA_494 | 72 | 72 | G | A |
| 107 | RIM2 | bRIM2_GT_149 | 72 | 72 | G | T |
| 108 | RIM2 | bRIM2_GT_99 | 72 | 72 | G | T |
| 109 | RIM2 | bRIM2_TC_230 | 72 | 72 | T | C |
| 110 | RIM2 | bRIM2_TC_360 | 72 | 72 | T | C |
| 111 | RIM2 | bRIM2_TG_472 | 72 | 72 | T | G |
| 112 | RIM2 | bRIM2_TG_667 | 72 | 72 | T | G |
| 113 | TLE4 | bTLE4_A988G | 72 | 72 | A | G |
| 114 | TLE4 | bTLE4_AC_108 | 72 | 72 | A | C |
| 115 | TLE4 | bTLE4_AC_114 | 72 | 72 | A | C |
| 116 | TLE4 | bTLE4_AG_134 | 72 | 72 | A | G |
| 117 | TLE4 | bTLE4_AG_161 | 72 | 72 | A | G |
| 118 | TLE4 | bTLE4_AG_212 | 72 | 72 | A | G |
| 119 | TLE4 | bTLE4_AG_260 | 72 | 72 | A | G |
| 120 | TLE4 | bTLE4_AG_307 | 72 | 72 | A | G |
| 121 | TLE4 | bTLE4_AG_458 | 72 | 72 | A | G |
| 122 | TLE4 | bTLE4_AG_89 | 72 | 72 | A | G |
| 123 | TLE4 | bTLE4_AT_152 | 72 | 72 | A | T |
| 124 | TLE4 | bTLE4_AT_262 | 72 | 72 | A | T |
| 125 | TLE4 | bTLE4_C1072T | 72 | 72 | C | T |
| 126 | TLE4 | bTLE4_C453T | 72 | 72 | C | T |
| 127 | TLE4 | bTLE4_C563A | 72 | 72 | C | A |
| 128 | TLE4 | bTLE4_CG_116 | 72 | 72 | C | G |
| 129 | TLE4 | bTLE4_CG_414 | 72 | 72 | C | G |
| 130 | TLE4 | bTLE4_CT_167 | 72 | 72 | C | T |
| 131 | TLE4 | bTLE4_CT_480 | 72 | 72 | C | T |
| 132 | TLE4 | bTLE4_CT_627 | 72 | 72 | C | T |
| 133 | TLE4 | bTLE4_CT_96 | 72 | 72 | C | T |
| 134 | TLE4 | bTLE4_D296_2 | 72 | 73 | CT | — |
| 135 | TLE4 | bTLE4_D393_1 | 72 | 72 | C | — |
| 136 | TLE4 | bTLE4_D615_2 | 72 | 73 | TT | — |
| 137 | TLE4 | bTLE4_G358T | 72 | 72 | G | T |
| 138 | TLE4 | bTLE4_G560A | 72 | 72 | G | A |
| 139 | TLE4 | bTLE4_G611A | 72 | 72 | G | A |
| 140 | TLE4 | bTLE4_G750A | 72 | 72 | G | A |
| 141 | TLE4 | bTLE4_G848A | 72 | 72 | G | A |
| 142 | TLE4 | bTLE4_G913C | 72 | 72 | G | C |
| 143 | TLE4 | bTLE4_GA_102 | 72 | 72 | G | A |
| 144 | TLE4 | bTLE4_GA_107 | 72 | 72 | G | A |
| 145 | TLE4 | bTLE4_GA_205 | 72 | 72 | G | A |
| 146 | TLE4 | bTLE4_GA_568 | 72 | 72 | G | A |
| 147 | TLE4 | bTLE4_GA_66 | 72 | 72 | G | A |
| 148 | TLE4 | bTLE4_GC_199 | 72 | 72 | G | C |
| 149 | TLE4 | bTLE4_GC_374 | 72 | 72 | G | C |
| 150 | TLE4 | bTLE4_GT_248 | 72 | 72 | G | T |
| 151 | TLE4 | bTLE4_GT_365 | 72 | 72 | G | T |
| 152 | TLE4 | bTLE4_GT_382 | 72 | 72 | G | T |
| 153 | TLE4 | bTLE4_I51_7 | 72 | 78 | TAACTTT | — |
| 154 | TLE4 | bTLE4_T1215C | 72 | 72 | T | C |
| 155 | TLE4 | bTLE4_T475C | 72 | 72 | T | C |
| 156 | TLE4 | bTLE4_TA_141 | 72 | 72 | T | A |
| 157 | TLE4 | bTLE4_TA_247 | 72 | 72 | T | A |
| 158 | TLE4 | bTLE4_TA_291 | 72 | 72 | T | A |
| 159 | TLE4 | bTLE4_TA_328 | 72 | 72 | T | A |
| 160 | TLE4 | bTLE4_TC_149 | 72 | 72 | T | C |
| 161 | TLE4 | bTLE4_TC_198 | 72 | 72 | T | C |
| 162 | TLE4 | bTLE4_TC_200 | 72 | 72 | T | C |
| 163 | TLE4 | bTLE4_TC_276 | 72 | 72 | T | C |
| 164 | TLE4 | bTLE4_TC_315 | 72 | 72 | T | C |
| 165 | TLE4 | bTLE4_TC_319 | 72 | 72 | T | C |
| 166 | TLE4 | bTLE4_TC_353 | 72 | 72 | T | C |
| 167 | TLE4 | bTLE4_TC_423 | 72 | 72 | T | C |
| 168 | TLE4 | bTLE4_TC_79 | 72 | 72 | T | C |
| 169 | TLE4 | bTLE4_TG_132 | 72 | 72 | T | G |
| 170 | TLE4 | bTLE4_TG_251 | 72 | 72 | T | G |
| 171 | TLE4 | bTLE4_TG_571 | 72 | 72 | T | G |

### REFERENCES

The references cited in this application, both above and below, are specifically incorporated herein by reference.

### Non-Patent Literature

Abdel-Azim, G and Freeman, A E, (2002) J. Dairy Sci. 85:1869-1880.
Blott, S., Kim, J. J., Moisio, S., et al. (2003). Genetics 163: 253-266.
Ciobanu, D C, Bastiaansen, J W M, Longergan, S M, Thomsen, H, Dekkers, J C M, Plastow, G S, and Rothschild, M F, (2004) J. Anim. Sci. 82:2829-39.
Cohen-Zinder, M. et al. (2005) Genome Res. 15:936-44.
Davis, G P and DeNise, S K, (1998) J. Anim. Sci. 76:2331-39.
Dekkers, J C M, and Chakraborty, R, (2001) J. Anim. Sci. 79:2975-90.
Demars J, Riquet J, Feve K, Gautier M, Morisson M, Demeure O, Renard C, Chardon P, Milan D. (2006), BMC Genomics, 24:7-13
Du and Hoeschele, (2000) Genetics 156:2051-62.
Ducrocq, V. 1987. An analysis of length of productive life in dairy cattle. Ph.D. Diss. Cornell Univ., Ithaca, N.Y.; Univ. Microfilms Int., Ann Arbor, Mich.
Everts-van der Wind A, Larkin D M, Green C A, Elliott J S, Olmstead C A, Chiu R, Schein J E, Marra M A, Womack J E, Lewin H A. (2005) Proc Natl Acad Sci USA, 20; 102(51):18526-31.
Falconer, D S, and Mackay, T F C, (1996) Introduction to Quantitative Genetics. Harlow, UK: Longman.
Fernando, R, and Grossman, M, (1989) Marker assisted selection using best linear unbiased prediction. Genetics Selection Evolution 21:467-77.
Franco, M M, Antunes, R C, Silva, H D, and Goulart, L R (2005) J. Appl. Genet. 46(2): 195-200.
Grisart, B. et al. (2002) Genome Res. 12:222-231
Grosz, M D, Womack, J E, and Skow, L C (1992) Genomics, 14(4):863-868.
Hayes, B, and Goddard, M E, (2001) Genet. Sel. Evol. 33:209-229.
Hayes, B, Chamberlain, A. J., Goddard, M. E. (2006) Proc. 8^{th} WCGALP 22:(16).
Kaminski, S, Ahman, A, Ruse, A, Wojcik, E, and Malewski, T (2005) J. Appl. Genet. 46(1):45-58.
Kaminski S, Brym P, Ruse A, and Wojcik E, (2006) Associations between Milk Performance Traits in Holstein Cows and 16 Candidate SNPs Identified by Arrayed Primer Extension (APEX) Microarray, Animal Biotechnology, 17:1-11, 2006.
Kuhn, C. et al. (2004). Genetics 167:1873-81.
Kwok P Y, Methods for genotyping single nucleotide polymorphisms, (2001), Annu. Rev. Genomics Hum. Genet., 2:235-258.
Meuwissen, T H E, and Van Arendonk, J A M, (1992) J. Dairy Sci. 75:1651-1659.
Meuwissen, T H E, Hayes, B J, and Goddard, M E, (2001) Genetics. 157:1819-29.
Rothschild and Plastow, (1999), Ag Bio Tech Net 10:1-8.
Schaeffer, L R (2006) J. Anim. Breed. Genet. 123:218-223.
Schnabel, R. et al. (2005) PNAS 102:6896-6901.
Schrooten, C, Bovenhuis, H, van Arendonk, J A M, and Bijma, P (2005) J. Dairy Sci. 88:1569-1581.
Sharma, B S, Jansen, G B, Karrow, N A, Kelton, D, and Jiang, Z, (2006) J. Dairy Sci. 89:3653-3663.
Short, T H, et al. (1997) J. Anim. Sci. 75:3138-3142.
Spelman, R J and Bovenhuis, H, (1998) Animal Genetics, 29:77-84.
Spelman, R J and Garrick, D J, (1998) J. Dairy Sci, 81:2942-2950.
Stearns, T M, Beever, J E, Southey, B R, Ellis, M, McKeith, F K and Rodriguez-Zas, S L, (2005) J. Anim. Sci. 83:1481-93.
Syvänen A C, Accessing genetic variation: genotyping single nucleotide polymorphisms, (2001) Nat. Rev. Genet. 2:930-942.
VanRaden, P. M. and E. J. H. Klaaskate. 1993. J. Dairy Sci. 76:2758-2764.
Verrier, E, (2001) Genet. Sel. Evol. 33:17-38.
Villanueva, B, Pong-Wong, R, Fernandez, J, and Toro, M A (2005) J. Anim. Sci. 83:1747-52.
Weller J I, Kashi Y, Soller M. (1990) J. Dairy Sci. 73:2525-37
Williams, J L, (2005), Rev. Sci. Tech. Off. Int. Epiz. 24(1):379-391.
Windig, J J, and Meuwissen, T H E, (2004) J. Anim. Breed. Genet. 121:26-39.
Winter, A. et al. (2002). PNAS, 99:9300-9305.
Womack, J, (1987), Dev. Genet. 8(4):281-293.
Yasue H, Kiuchi S, Hiraiwa H, Ozawa A, Hayashi T, (2006), Cytogenet. Genome Res., 112(1-2):121-125.
Youngerman, S M, Saxton, A M, Oliver, S P, and Pighetti, G M, (2004) J. Dairy Sci. 87:2442-2448.

Patent Literature (Dairy)

| Patent/Publication Number | Title | Inventors | Pub. Date |
|---|---|---|---|
| U.S. Pat. No. 5,041,371 | Genetic marker for superior | Cowan, Charles M.; Dentine, Margaret | Aug. 20, 1991 |
| | milk products in dairy cattle | R.; Ax, Roy L.; Schuler, Linda A. | |
| U.S. Pat. No. 5,374,523 | Allelic variants of bovine | Collier, Robert J.; Mauser, Scott D.; | Dec. 20, 1994 |
| | somatotropin gene: genetic | Krivi, Gwen G.; Lucy, Matthew C. | |
| | marker for superior milk | | |
| | production in bovine | | |
| U.S. Pat. No. 5,582,987 | Methods for testing bovine for | Lewin, Harris A.; van Eijk, Michiel J. T. | Dec. 10, 1996 |
| | resistance or susceptibility to | | |
| | persistent lymphocytosis by | | |
| | detecting polymorphism in | | |
| | bola-dr3 exon 2 | | |
| U.S. Pat. No. 5,614,364 | Genetic marker for improved | Tuggle, Christopher K.; Freeman, Albert E. | Mar. 25, 1997 |
| | milk production traits in cattle | | |
| US2003162207A1 | Multi-gene tests with ROC plots | Comings, David E.\|MacMurray, James P. | Aug. 28, 2003 |
| | for the assessment of risk for | | |
| | polygenic disorders | | |
| US2003039737A1 | Population of dairy cows | Cooper, Garth J. S. | Feb. 27, 2003 |
| | producing milk with desirable | | |
| | characteristics and methods of | | |
| | making and using same | | |
| US2004076977A1 | Marker assisted selection of | Georges, Michel Alphonse Julien; | Apr. 22, 2004 |
| | bovine for improved milk | Coppieters, Wonter Herman Robert; Grisart, | |
| | production using diacylglycerol | Bernard Marie-Josee Jean; Shell, Russell | |
| | acyltransferase gene dgat1 | Grant; Jean Reid, Suzanne; Ford, Christine | |
| | | Ann; Spelman, Richard John | |
| US2004115701A1 | Method for risk assessment | Comings, David E.\|MacMurray, | Jun. 17, 2004 |
| | for polygenic disorders | James P. | |
| US2004234986A1 | Method of testing a mammal for | Fries, Hans-Rudolf; Winter, Andreas | Nov. 25, 2004 |
| | its predisposition for fat content | | |
| | of milk and/or its | | |
| | predisposition for meat marbling | | |
| US2004241723A1 | Systems and methods for | Marquess, Foley Leigh Shaw; Laarveld, | Dec. 2, 2004 |
| | improving protein and milk | Bernard; Cleverly Buchanan, Fiona; Van | |
| | production of dairy herds | Kessel, Andrew Gerald; Schmutz, Sheila | |
| | | Marie; Waldner, Cheryl; Christensen, David | |
| US2004254104A1 | Marker assisted selection of | Blott, Sarah; Kim, Jong-Joo; Schmidt- | Dec. 16, 2004 |
| | bovine for improved milk | Kuntzel, Anne; Cornet, Anne; Berzi, | |
| | composition | Paulette; Cambisano, Nadine; Grisart, | |
| | | Bernard; Karim, Latifa; Simon, Patricia; | |
| | | Georges, Michel; Farnir, Frederic; | |
| | | Coppieters, Wouter; Moisio, Sirja; Vilkki, | |
| | | Johanna; Spelman, Richard; Johnson, Dave; | |
| | | Ford, Christine; Snell, Russell | |
| US2005015827A1 | QTL “mapping as-you-go” | Podlich, Dean\|Cooper, | Jan. 20, 2005 |
| | | Mark\|Winkler, Chris | |
| US2005123929A1 | Methods and compositions for | Khatib, Hasan | Jun. 9, 2005 |
| | genetically detecting improved | | |
| | milk production traits in cattle | | |
| US2005136440A1 | Method for identifying animals | Renaville, Robert; Gengler, Nicolas | Jun. 23, 2005 |
| | for milk production qualities by | | |
| | analysing the polymorphism of | | |
| | the pit-1 and kappa-casein genes | | |
| US2005137805A1 | Gene expression profiles that | Lewin, Harris A.; Liu, Zonglin; | Jun. 23, 2005 |
| | identify genetically elite | Rodriguez-Zas, Sandra; Everts, Robin E. | |
| | ungulate mammals | | |
| US2005153317A1 | Methods and systems for | DeNise, Sue; Rosenfeld, David; Kerr, | Jul. 14, 2005 |
| | inferring traits to breed and | Richard; Bates, Stephen; Holm, Tom | |
| | manage non-beef livestock | | |
| US2006037090A1 | Selecting animals for desired | Andersson, Leif; Andersson, Goran; | Feb. 16, 2006 |
| | genotypic or potential | Georges, Michel; Buys, Nadine | |
| | phenotypic properties | | |
| US2006094011A1 | Method for altering fatty acid | Morris, Christopher Anthony; Tate, | May 4, 2006 |
| | composition of milk | Michael Lewis | |
| US2006121472A1 | Method for determining the | Prinzenberg, Eva-Maria; Erhardt, George | Jun. 8, 2006 |
| | allelic state of the 5′-end of the | | |
| | $g(a)s1- casein gene | | |
| US2006166244A1 | Dna markers for increased milk | Schnabel, Robert D.; Sonstegard, Tad | Jul. 27, 2006 |
| | production in cattle | S.; Van Tassell, Curtis P.; Ashwell, | |
| | | Melissa S.; Taylor, Jeremy F. | |
| US2007026493A1 | System and method for | Paszek; Adam A.\|Burghardi; Steve | Oct. 10, 2002 |
| | optimizing animal production | R.\|Cook; David A.\|Engelke; | |
| | using genotype information | Gregory L.\|Giesting; Donald | |
| | | W.\|Knudson; Brian J.\|McGoogan; | |
| | | Bruce B.\|Messman; Michael | |
| | | A.\|Newcomb; Mark D.\|van | |
| | | de Ligt; Jennifer L. G. | |
| WO 02/080079A2 | System and Method for the | Balmain, Alan; Healey, Lee Anne; Reijerse, | Oct. 10, 2002 |
| | Detection of Genetic | Fidel | |
| | Interactions in Complex Trait | | |
| | Diseases | | |
| WO0236824A1 | Marker assisted selection of | GEORGES, MICHEL, ALPHONSE, JULIEN; | May 10, 2002 |
| | bovine for improved milk | COPPIETERS, WOUTER, HERMAN, ROBERT; | |
| | production using diacylglycerol | GRISART, BERNARD, MARIE-JOSEE, | |
| | acyltransferase gene dgat1 | JEAN; SNELL, RUSSELL, GRANT; REID, | |
| | | SUZANNE, JEAN; FORD, CHRISTINE, | |
| | | ANN; SPELMAN, RICHARD, JOHN | |
| WO03104492A1 | Marker assisted selection of | BLOTT, SARAH; KIM, JONG-JOO; | Dec. 18, 2003 |
| | bovine for improved milk | SCHMIDT-KUNTZEL, ANNE; CORNET, | |
| | composition | ANNE; BERZI, PAULETTE; CAMBISANO, | |
| | | NADINE; GRISART, BERNARD; KARIM, | |
| | | LATIFA; SIMON, PATRICIA; GEORGES, | |
| | | MICHEL; FARNIR, FREDERIC; COPPIETERS, | |
| | | WOUTER; MOISIO, SIRJA; VILKKI, | |
| | | JOHANNA; JOHNSON, DAVE; SPELMAN, | |
| | | RICHARD; FORD, CHRISTINE; SNELL, | |
| | | RUSSELL | |
| WO04004450A1 | Method for altering fatty acid | MORRIS, Christopher Anthony; TATE, | Jan. 15, 2004 |
| | composition of milk | Michael Lewis | |
| WO04048609A2 | Methods and kits for the | RENAVILLE, Robert; PARMENTIER, | Jun. 10, 2004 |
| | selection of animals having | Isabelle | |
| | certain mild production | | |
| | capabilities, based on the | | |
| | analysis of a polymorphism in | | |
| | the somatotropin receptor gene | | |
| WO04083456A1 | Systems and methods for | MARQUESS, Foley, Leigh, Shaw; LAARVELD, | Sep. 30, 2004 |
| | improving protein and milk | Bernard; CLEVERLY BUCHANAN, Fiona; VAN | |
| | production of dairy herds | KESSEL, Andrew, Gerald; SCHMUTZ, | |
| | | Sheila, Marie; WALDNER, Cheryl; | |
| | | CHRISTENSEN, David | |
| WO05007881A2 | Improving production | SCHMUTZ, SHEILA MARIE; GOODALL, | Jan. 27, 2005 |
| | characteristics of cattle | JULIE JANINE | |
| WO05030789A1 | Adrenergic receptor snp for | COLLIER, Robert, J.; LOHIUS, | Apr. 7, 2005 |
| | improved milking characteristics | Michael; GROSZ, Michael | |
| WO05040400A2 | Methods and systems for | DENISE, Sue, K.; ROSENFELD, David; KERR, | May 6, 2005 |
| | inferring traits to manage non- | Richard; BATES, Stephen; HOLM, Tom | |
| | beef livestock | | |
| WO05056758A2 | Methods and compositions for | KHATIB, Hasan | Jun. 23, 2005 |
| | genetically detecting improved | | |
| | milk production traits in cattle | | |
| WO05089122A2 | Animals with reduced body fat | JOHNSON, Geoffrey, B.; PLATT, | Sep. 29, 2005 |
| | and increased bone density | Jeffrey, L.; JOHNSON, Joel, W. | |
| WO06076563A2 | Dna markers for increased milk | SCHNABEL, Robert, D.; SONSTEGARD, Tad, | Jul. 20, 2006 |
| | production in cattle | S.; VAN TASSELL, Curtis, P.; ASHWELL, | |
| | | Melissa, S.; TAYLOR, Jeremy, F. | |
| WO06094774A2 | REVERSE PROGENY | DIRKS, Robert, Helene, | Sep. 14, 2006 |
| | MAPPING | Ghislain\|SCHUT, Johannes, Wilhelmus | |
| WO9213102A1 | Polymorphic DNA markers in | Georges, Michel; MASSEY, Joseph, M. | Aug. 6, 1992 |
| | bovidae | | |
| WO9319204A1 | Bovine alleles and genetic | LEWIN, Harris, A.; VAN EIJK, Michiel, | Sep. 30, 1993 |
| | markers and methods of testing | J., T. | |
| | of and using same | | |
| WO9403641A1 | Genetic marker for dairy cattle | COLLIER, Robert, Joseph; HAUSER, | Feb. 17, 1994 |
| | production superiority | Scott, David; KRIVI, Gwen, Grabowski; | |
| | | LUCY, Matthew, Christian | |
| WO9849887A1 | SOYBEAN HAVING | LARK, Karl, G.\|ORF, | Nov. 12, 1998 |
| | EPISTATIC GENES | James\|CHASE, Kevin\|ADLER, Fred | |
| | AFFECTING YIELD | | |

1. A method for allocating one or more animals for use according to each animal's predicted marker breeding value for productivity and/or fitness, the method comprising:
a. evaluating one or more animals to determine each animal's genotype at one or more locus/loci; wherein at least one locus comprises a single nucleotide polymorphism (SNP) that has at least two allelic variants and that is selected from the SNPs described in Table 1;
b. analyzing the determined genotype of at least one evaluated animal, at one or more SNPs selected from the SNPs described in Table 1, to determine which allelic variant(s) is/are present;
c. associating said allelic variant(s) with at least one productivity or fitness trait as described in Table 1.
d. allocating the animal for use according to its determined genotype.

2. The method of claim 1 further wherein said analyzing further comprises an analysis of at least one interaction effect described in Table 1.

3. The method of claim 1 wherein the animal's genotype is evaluated at two or more loci that contain SNPs selected from the SNPs described in Table 1.

4. The method of claim 1 wherein the animal's genotype is evaluated at 10 or more loci.

5. The method of claim 1 wherein the animal's genotype is evaluated at 100 or more loci.

6. The method of claim 1 wherein the animal's genotype is evaluated at 200 or more loci.

7. The method of claim 1 wherein SNPs evaluated are associated with a fitness trait selected from the group consisting of pregnancy rate (PR), daughter pregnancy rate (DPR), productive life (PL), somatic cell count (SCC) and somatic cell score (SCS).

8. The method of claim 1 wherein SNPs evaluated are associated with a productivity trait selected from the group consisting of total milk yield, milk fat percentage, milk fat yield, milk protein percentage, milk protein yield, total lifetime production, milking speed and lactation persistency.

9. The method of claim 1 that comprises whole-genome analysis.

10. A method for selecting one or more potential parent animal(s) for breeding to improve fitness and/or productivity in potential offspring:
a. determining at least one potential parent animal's genotype at least one genomic locus; wherein at least one locus contains a single nucleotide polymorphism (SNP) that has at least two allelic variants and that is selected from the SNPs described in Table 1;
b. analyzing the determined genotype of at least one evaluated animal for one or more SNPs selected from the SNPs described in Table 1 to determine which allele is present;
c. correlating the identified allele with a fitness and/or productivity phenotype;
d. allocating at least one animal for breeding use based on its genotype.

11. The method of claim 10 wherein analyzing comprises at least one estimate of an interaction effect described in Table 1.

12. The method of claim 10 wherein the potential parent animal's genotype is evaluated at five or more loci that contain SNPs selected from the SNPs described in Table 1.

13. The method of claim 10 wherein the potential parent animal's genotype is evaluated at 10 or more loci, including at least two loci that contain SNPs selected from the SNPs described in Table 1.

14. The method of claim 10 wherein the potential parent animal's genotype is evaluated at 20 or more loci, including at least two loci that contain SNPs selected from the SNPs described in Table 1.

15. The method of claim 10 wherein the potential parent animal is selected to improve fitness in the potential offspring.

16. The method of claim 10 wherein the potential parent animal is selected to improve productivity in the potential offspring.

17. The method of claim 10 that comprises whole-genome analysis.

18-34. (canceled)
